(19) United States
(12) Patent Application Publication
(10) Pub. No.: US 2006/0019936 A1
(43) Pub. Date: Jan. 26, 2006

(54) Methods and compositions for treating cancer

(75) Inventors: John M. Eissigmann, Cambridge, MA (US); Robert G. Croy, Belmont, MA (US)

Correspondence Address: WOLF GREENFIELD & SACKS, PC;FEDERAL RESERVE PLAZA, 600 ATLANTIC AVENUE, BOSTON, MA, 02210-2211, US

(73) Assignee: Massachusetts Institute of Technology, Cambridge, MA (US)

(21) Appl. No.: 11/078,645

(22) Filed: Mar. 10, 2005

Related U.S. Application Data
(60) Provisional application No. 60/552,322, filed on Mar. 10, 2004.

(51) Int. Cl. A61K 31/573

(52) U.S. Cl. 514/179

(57) ABSTRACT

The invention provides compounds and methods for treating cancer. Exemplary compounds are multi-functional compounds with two different moieties connected by a linker. Compounds of the invention can activate one or more pathways that result in the inhibition of cell growth. The invention includes cytostatic and cytotoxic compounds. Methods and compositions of the invention are particularly useful for treating cancer cells that are resistant to other chemotherapeutic drugs.

### RELATED APPLICATIONS

This application claims benefit under 35 U.S.C. 119(e) of the filing date of U.S. Ser. No. 60/552,322 filed on Mar. 10, 2004, the entire disclosure of which is incorporated herein by reference.

### FEDERALLY SPONSORED RESEARCH

This invention was made with Government support under grant R01 CA077743 from the National Cancer Institute. The Government may have certain rights to this invention.

### BACKGROUND OF INVENTION

Cytotoxic agents that act by covalent modification of DNA were the first modern anticancer chemotherapeutics and remain major components of combination chemotherapy regimens. In combination with drugs that act by other mechanisms, alkylating antitumor drugs have produced impressive and even curative responses in the treatment of some cancers (e.g., cisplatin in testicular cancer). Frequently, however, tumors are found to have inherent resistance to these compounds or to develop resistance during the course of treatment. The rapid evolution of resistance makes it important to develop new agents that can defeat the molecular barriers responsible for clinical failure.

### SUMMARY OF THE INVENTION

Aspects of the invention provide methods and compositions (including cytotoxic and cytostatic compositions) useful for treating cancer and other diseases. In one aspect, cytotoxic compositions of the invention may be apoptosis inducing agents and may be useful to treat diseases or conditions that are currently treated with alkylating agents. In one aspect, embodiments of the invention are multifunctional compounds that disrupt multiple biochemical pathways responsible for tumor growth and survival. Certain compounds of the invention incorporate several mechanisms of action into a single anticancer agent.

In one aspect, compounds of the invention may include a bi-functional alkylating moiety. In another aspect, compounds of the invention may include a variant of the bi-functional alkylating moiety that is mono-substituted in that one of the alkylating arms of the bi-functional alkylating moiety is substituted with a non-alkylating group. In yet a further aspect, compounds of the invention may include a variant of the bi-functional alkylating moiety that is di-substituted in that both of the alkylating arms of the bi-functional alkylating moiety are substituted with a non-alkylating group(s).

According to aspects of the invention, the bi-functional alkylating moiety may be linked by a linker that is stable and/or soluble under intracellular conditions to a ligand that binds to one or more intracellular molecules (e.g., nucleic acid, lipid, or protein).

In one aspect, without wishing to be bound by theory, it is thought that compositions of the invention may damage and bind to cellular nucleic acid (e.g., genomic DNA) via an alkylating moiety and shield the damaged nucleic acid from repair due to the binding of the ligand to a specific intracellular molecule. Alternatively, or in addition, without wishing to be bound by theory, it is thought that compositions of the invention may act as “sinks” by binding a specific important intracellular molecule thereby decreasing its effective intracellular concentration. However, Applicants have found surprisingly that certain compounds of the invention are cytotoxic and induce apoptosis in diseased cells (e.g., cancer cells) that do not express or over-express the intracellular molecule (e.g., a receptor) that the ligand is known to bind to. In addition, Applicants have found that certain compounds that contain a mono- or di-substituted bi-functional alkylating moiety may be cytostatic and/or cause cell cycle arrest.

Accordingly, in one aspect the invention provides a method for killing androgen receptor negative cells by contacting the cells with an effective amount of a compound that includes a bifunctional DNA damaging moiety that is linked by a linker stable under intracellular conditions to a ligand for an androgen receptor. The androgen may be testosterone (e.g., dihydroxy-testosterone). In one embodiment, the ligand may be estradienone. In one embodiment, the compound is 11β-{N-(2-(N-((N,N-bis-2-chloroethylaminophenyl)propyl)-carbamoyloxy) ethyl)aminohexyl}-17β-hydroxy-estra-Δ4(5),9(10)-3-one. In one embodiment, the androgen receptor negative cells are cancer cells. The cancer cells may be breast, ovarian, endometrial, colon, melanoma, lymphoma and/or pancreatic cancer cells.

In another aspect, the invention provides a method for killing estrogen receptor negative cells by contacting the cells with an effective amount of a compound that includes a bifunctional DNA damaging moiety that is linked by a linker that is stable under intracellular conditions to a ligand for an estrogen receptor. The estrogen may be progesterone. In one embodiment, the ligand may be 2-phenyl-indole. In one embodiment, the ligand may be estradiol. In one embodiment, the compound is 1-6{N-[2-(N-((N,N-bis-2-chloroethylaminophenyl)propyl)-carbamoyl oxy)ethyl]aminohexyl}-5-hydroxy-2-(4-hydroxyphenyl)-3-methyl indole. In one embodiment, the compound is 7α-{N-(2-(N-((N,N-bis-2-chloroethylaminophenyl)propyl)-carbamoyloxy) ethyl)aminohexyl}-3,17β-dihydroxyestra-1,3,5(10)-triene. In one embodiment, the estrogen receptor negative cells are cancer cells. The cancer cells may be prostate, colon, melanoma, lymphoma and/or pancreatic cancer cells.

In yet another aspect, the invention provides a method for killing vitamin D receptor negative cells by contacting the cells with an effective amount of a compound that includes a bifunctional DNA damaging moiety that is linked by a linker that is stable under intracellular conditions to a ligand for a vitamin D₃ receptor. In one embodiment, the ligand may be vitamin D₃. In one embodiment, in the compound may be 11β-{N-(2-(N-((N,N-bis-2-chloroethylaminophenyl)propyl)-carbamoyloxy) ethyl)aminohexyl}-17β-hydroxy-estra-Δ4(5),9(10)-3-one. In one embodiment, the compound may be (3-{4-[Bis-(2-chloro-ethyl)-amino]-phenyl}-propyl)-carbamic acid 2-[3-(4-{4-[2-(3,5-dihydroxy-2-methylene-cyclohexylidene)-ethylidene]-7α-methyl-octahydro-inden-yl}-8-hydroxy-8-methyl-nonyloxy)-propylamino]-ethyl ester. In one embodiment, the cells may be cancer cells. The cancer cells may be breast, ovarian, lymphoma and/or endometrial cancer cells.

In another aspect, the invention provides a cell membrane permeant compound that is effective in inducing cell cycle arrest. In one embodiment, the compound includes a non-alkylating variant of a bi-functional alkylating moiety (wherein both alkylating groups are substituted with a non-alkylating group) linked by a linker stable under intracellular conditions to an agent that mediates binding of a cellular protein to the compound. The compound may include a non-alkylating aniline moiety. The agent may be a ligand for a steroid or secosteroid receptor. The ligand may be estradienone, estradiol, 2-phenylindole, vitamin D₃, or any other suitable ligand.

In another embodiment, the compound that is effective in inducing cell-cycle arrest may include a variant of a bi-functional alkylating moiety that is monofunctional alkylating moiety (wherein one of the alkylating groups on the bi-functional alkylating moiety is substituted with a non-alkylating group) linked by a linker stable under intracellular conditions to an agent that mediates binding of a cellular protein to the compound. The monofunctional alkylating moiety may be a monofunctional aniline moiety. The agent may be a ligand for a steroid or secosteroid receptor. The ligand may be estradienone, estradiol, 2-phenylindole, vitamin D₃, or any other suitable ligand.

In one aspect, the invention provides methods for inducing cell cycle arrest by contacting a target cell (e.g., a cancer or other diseased cell) with a sufficient amount of one or more compounds that are effective to induce cell cycle arrest.

In yet a further aspect, the invention provides methods for treating cancer by administering to a cancer patient one or more compounds that are effective for inducing cell-cycle arrest along with one or more other anti-cancer agents.

In one embodiment, the invention provides a method for treating a patient with an androgen receptor negative cancer by administering to the patient a therapeutically effective amount of a compound that includes a bifunctional DNA damaging moiety that is linked by a linker stable under intracellular conditions to a ligand for an androgen receptor (e.g., for a testosterone receptor, for example a dihydroxytestosterone receptor).

In another embodiment, the invention provides a method for treating a patient with an estrogen receptor negative cancer by administering to the patient a therapeutically effective amount of a compound that includes a bifunctional DNA damaging moiety that is linked by a linker stable under intracellular conditions to a ligand for an estrogen receptor (e.g., for a progesterone receptor).

In yet another embodiment, the invention provides a method for treating a patient with a vitamin D receptor negative cancer by administering to the patient a therapeutically effective amount of a compound that includes a bifunctional DNA damaging moiety that is linked by a linker stable under intracellular conditions to a ligand for a vitamin D receptor.

Useful compounds of the invention include compounds shown in FIG. 1 and FIG. 5.

In another aspect, the invention provides a method for treating a Skp2 over-expressing cancer. In one embodiment, a cancer is treated by determining the level of Skp2 expression in the cancer (e.g., in a cancer cell or a cancer tissue biopsy) and if the level of Skp2 expression (e.g., RNA and/or protein level) is above a reference level (e.g., a normal level in a normal cell) contacting the cancer cells with an effective amount of a compound of the invention that contains a bifunctional DNA damaging moiety linked by a linker that is stable under intracellular conditions to a ligand for a steroid or secosteroid receptor.

In another aspect, the invention provides a method for treating cancer a Myc over-expressing cancer. In one embodiment, a cancer is treated by determining the level of Myc expression in the cancer (e.g., in a cancer cell or a cancer tissue biopsy) and if the level of Myc expression (e.g., RNA and/or protein level) is above a reference level (e.g., a normal level in a normal cell) contacting the cancer cells with an effective amount of a compound of the invention that contains a bifunctional DNA damaging moiety linked by a linker that is stable under intracellular conditions to a ligand for a steroid or secosteroid receptor.

In another aspect, the invention provides a method for treating a Bcl-2 over-expressing cancer. In one embodiment, a cancer is treated by determining the level of Bcl-2 expression in the cancer (e.g., in a cancer cell or a cancer tissue biopsy) and if the level of Bcl-2 expression (e.g., RNA and/or protein level) is above a reference level (e.g., a normal level in a normal cell) contacting the cancer cells with an effective amount of a compound of the invention that contains a bifunctional DNA damaging moiety linked by a linker that is stable under intracellular conditions to a ligand for a steroid or secosteroid receptor.

Similarly, aspects of the invention are useful for treating Bcl-xl over-expressing cancers and cancers that over-express one or more other members of the Bcl family of genes that have been associated with chemotherapy resistance (e.g., resistance to therapeutic alkylating agents). Similarly, aspects of the invention are useful for treating cancers that over-express one or more other IAP (Inhibitor of Apoptosis) family members that lead to chemotherapy resistance (e.g., resistance to therapeutic alkylating agents).

In another aspect the invention provides a method for treating cancer with mutated cellualr proteins e.g. tumor suppressors such as p53, oncogenes such as k-Ras etc.

In another aspect, the invention provides a method for treating a cancer with an abnormally high level of p70S6K activity. In one embodiment, a cancer is treated by determining the level of phosphorylation of p70S6K in the cancer (e.g., in a cancer cell or a cancer tissue biopsy) and if the level of phosphorylation of p70S6K is above a reference level (e.g., a normal level in a normal cell) contacting the cancer cells with an effective amount of a compound that contains a bifunctional DNA damaging moiety linked by a linker that is stable under intracellular conditions to a ligand for a steroid or secosteroid receptor.

Similarly, methods of the invention are useful for treating other cancers associated with an abnormal expression level of a protein or RNA or an abnormal level of protein activity (e.g., of a phosphorylated protein, for example TOR), wherein compounds of the invention are shown to decrease the expression level or the level of the active protein (e.g., the phosphorylated protein, for example TOR).

In one aspect of the invention, the biological and/or therapeutic effectiveness of alkylating agents (e.g., bifunctional alkylating agents) may be increased by linking the alkylating agent via a linker stable and/or soluble under intracellular conditions to a ligand that binds to one or more intracellular molecules.

Accordingly, compounds of the invention that include bi-functional alkylating moieties may be used to kill cells that are resistant to standard nucleic acid damaging agents.

In any of the methods described herein, the cells may be contacted in vivo by administering a compound of the invention to a subject that has cancer or other disease. Accordingly, aspects of the invention may include treating patients having one or more cancers or other diseases by administering a therapeutically effective amount of one or more compounds of the invention. Aspects of the invention also may be useful for treating metastatic cancers.

It should be appreciated that compounds of the invention may be provided in a pharmaceutical composition and also in a stereoisomeric form or a pharmaceutically acceptable acid or base addition salt form thereof.

The linker of any of the compounds described herein may include an alkyl-amino-carbamate alkyl chain. In one embodiment, the alkyl chain may have six carbons.

### BRIEF DESCRIPTION OF DRAWINGS

The accompanying drawings, are not intended to be drawn to scale. In the drawings, each identical or nearly identical component that is illustrated in various figures is represented by a like numeral. For purposes of clarity, not every component may be labeled in every drawing. In the drawings:

FIG. 1 A and B show embodiments of compounds of the invention;

FIG. 2 A-D shows embodiments of synthetic methods of the invention;

FIG. 3 shows an embodiment of a compound of the invention that is useful to kill androgen receptor negative cells;

FIG. 4 shows the inhibition of human LNCaP cell growth in mice using the 11β-dichloro compound;

FIG. 5 shows embodiments of 11β compounds of the invention that are useful for inducing cell cycle arrest;

FIG. 6 shows LNCaP cell morphology and cell cycle analysis of LNCaP cells treated with 11β compounds;

FIG. 7 shows immunoblot analysis of cell cycle checkpoint proteins in LNCaP cells treated with 11β compounds;

FIG. 8 shows activation of apoptosis markers in LNCaP cells induced by an 11β compounds;

FIG. 9 shows protein level changes induced by an 11β-dichloro compound;

FIG. 10 shows protein level changes induced by chlorambucil;

FIG. 11 shows protein level changes induced by an 11β-dimethoxy compound;

FIG. 12 shows compounds of the invention comprising varying linkers;

FIG. 13 shows A) structure and molecular features of a compound of the invention, B) survival of MCF-7 (ER+) and MDA-MB231 (ER−) breast cancer cells treated with estradiol compounds of the invention; and

FIG. 14 shows embodiments of synthetic methods of the invention.

### DETAILED DESCRIPTION

The invention provides methods and compositions for treating cancer. Compounds of the invention can inhibit DNA repair pathways; induce apoptosis; and/or cause cell cycle arrest.

Compounds of the invention are multi-functional compounds with at least two different moieties linked via a linker that is stable and/or soluble under intracellular conditions. In one aspect, a compound contains a first moiety that is reactive with nucleic acid such as cellular DNA (e.g., genomic DNA). In one embodiment, the first moiety contains a bifunctional alkylating moiety (e.g., a bifunctional aniline moiety). Ih another aspect of the invention, the first moiety contains a variant of the bifunctional alkylating moiety that is monosubstituted or disubstituted such that one or both of the alkylating groups of the bifunctional alkylating moiety are replaced with a non-alkylating group. In one embodiment, both alkylating groups are replaced with the same non-alkylating group. In another embodiment, each alkylating group is replaced with a different non-alkylating group. A non-alkylating group may be an alkyl, e.g. a mehtoxy etc. According to the invention, the first moiety is connected via the linker to a ligand that binds (e.g., with high affinity, for example in micromolar or nanomolar amounts) to one or more intracellular molecules (e.g., one or more proteins, nucleic acids, and/or lipids). In one embodiment, the ligand may be a protein recognition moiety. Accordingly, certain compounds of the invention are more alkylating (and more reactive with DNA) than other compounds. Depending on the configuration of the moiety that can be reactive with DNA, the compounds of the invention can be in one of the following embodiments: compounds that can form bifunctional adducts with nucleic acids, compounds that can form monofunctional adducts with nucleic acids and compounds that can not form nucleic acid adducts at all. As discussed herein, these different types of compounds have different types of effects on cells. According to the invention, compounds that can form bifunctional adducts can induce apoptosis and cell death. In contrast, compounds that can form monofunctional adducts can induce cell cycle arrest.

Accordingly, in certain embodiments of the invention, a compound contains an aniline moiety that can form bifunctional adducts with DNA. In other embodiments, a compound contains an aniline moiety that can only form monofunctional adducts with DNA. In yet other embodiments, the aniline moiety is di-substituted and can not react with DNA at all.

In certain embodiments of the invention, the ligand may be a protein recognition moiety that binds to a cellular protein (e.g., such as a steroid receptor, a kinase, a DNA repair protein, and/or a nuclear protein).

In one aspect, compounds of the invention may be multifunctional agents that include i) a steroid receptor ligand domain, ii) a nitrogen mustard domain (that can be inactivated) and iii) a linker that is soluble and stable under intracellular conditions.

Compounds of the invention are useful for treating cancer. In one embodiment, compounds of the invention are useful for treating cancer that over-expresses a cancer-specific protein such as a receptor (e.g., an androgen receptor, an estrogen receptor, a testosterone receptor, a progesterone receptor, etc., or any combination thereof). However, compounds of the invention also are useful for treating cancers that do not express or do not over-express a specific receptor such as a steroid receptor (e.g., an androgen receptor, an estrogen receptor, a testosterone receptor, a progesterone receptor, etc., or any combination thereof). Such cancers may be certain breast, prostate, liver, testicular, lung, colon, pancreatic, and/or ovarian cancers etc.

In another aspect, compounds of the invention (particularly apoptosis inducing compounds) are useful for treating cancers that are resistant to chemotherapy (e.g., resistant to alkylating agents such as DNA damaging compounds). In certain embodiments, compounds of the invention (particularly apoptosis inducing compounds) are useful for treating cancers that are resistant to other treatments due to the expression of one or more anti-apoptotic factors (e.g. Bcl-2 and/or Bcl-xl expressing cancers or tumors, or cancers or tumors that express/over-express one or more other Bcl or IAP family members that are associated with resistance to chemotherapy) or the activation of other survival mechanisms (e.g. mutation of p10), including mechanisms of apoptosis avoidance or apoptotic resistance. Compounds of the invention are particularly useful for treating prostate cancer that is refractory to treatment with conventional cytotoxic therapies as well as advanced metastatic disease that is resistant to hormonal antagonists.

Methods of the invention include contacting one or more cancer cells with a therapeutically effective amount of a compound or composition of the invention. The contacting can be in cultured cells, ex vivo cells or tissue, or in vivo depending on the application.

Compounds and methods of the invention are described in more detail in the following sections.

Compounds

Aspects of the invention provide compounds that are useful for treating cancer and/or other diseases. In one embodiment, alkylating compounds of the invention are useful for treating diseases that are responsive to alkylating agents. In general, compounds of the invention comprise a ligand that interacts with an intracellular molecule such as a receptor (e.g. an estrogen receptor (ER) or an androgen receptor (AR)) linked via a linker that is soluble and stable under intracellular conditions to i) a reactive first moiety that can covalently react with nucleophilic sites in DNA or other cellular molecules or ii) a less reactive or non-reactive variant of the reactive first moiety.

Ligands that Interact with Intracellular Receptors

A compound of the invention may include one or more ligands that interact with intracellular molecules. A ligand is preferably a small organic molecule that binds with greater than micromolar affinity (e.g., with high affinity) to a protein. Accordingly, A ligand may interact with one or more proteins, including, for example, a nuclear protein, a cytoplasmic protein, and/or a membrane bound protein. The target protein may be, for example, a kinase, a receptor (e.g., a steroid receptor, a glucocorticoid receptor, an androgen receptor, an estrogen receptor, progesterone receptor, a testosterone receptor, a dihydroxytestosterone receptor, or another specific receptor, or a combination thereof), or a DNA repair protein, etc., or any combination thereof. Accordingly, the ligand may be a steroid (e.g., an androgen receptor binding steroid, or an estrogen receptor binding steroid, progesterone, testosterone, or an analog thereof, etc.). In one embodiment, the steroid ligand may estradiol, 2-phenylindole, estradienone, 4-hydroxytamoxifen, ICI 182,780, dihydrotestosterone, testosterone, dexamethasone, mifepristone, progesterone, cortisol, coumestrol, PPT, DPN, genistein, androstane, bufanolide, campestane, cardanolide, cholane, cholestane, ergostane, estrane, furostan, gonane, gorgostane, poriferastane, pregnane, spirostanstigmastane, cholesterol, vitamin D₃, vitamin D₂, or an analog or derivative of any one of the above. In certain embodiments, a ligand may be a substrate or substrate analog (e.g., ATP or an ATP analog that may bind to a kinase). In other embodiments, a ligand may bind one or more orphan receptors (e.g., one or more orphan receptors that are specifically over-expressed in a cancer or other diseased cell).

Moieties that can Covalently React with Nucleophilic Sites in Nucleic Acids and Variants Thereof

In one aspect, a compound of the invention may include one or more reactive moieties that can covalently react with nucleophilic sites in nucleic acids (e.g., DNA such as genomic DNA) or other intracellular molecules. Each moiety may be a bi-functional moiety in that it may have two arms, each of which may contain a reactive group. Such a moiety may be any DNA alkylating moiety that is capable of forming bifunctional DNA adducts, such as a bifunctional aniline moiety. In one embodiment, the moiety is a nitrogen mustard. In another aspect of the invention, a compound may contain a moiety that is a less-reactive or a non-reactive variant of a bifunctional reactive moiety in that one or both of the reactive groups may be substitute with a less reactive or non-reactive group.

Accordingly, a reactive moiety of the invention may contain one or more of the following alkylating moieties: chloroethylnitrosourea, alkylsulfonate, hexamethylmelamine, triethylenemelamine, aziridine, antineoplastic antibiotic or nitrogen mustard. Chloroethylnitrosourea moieties, or analogs or derivatives thereof, may belong to a group including, but not limited to, carmustine, chlorozoticin, lomustine, nimustine, ranimustine, streptozotocin, an aniline moiety that forms bifunctional adducts with DNA, such as a nitrogen mustard compound. An alkylsulfonates may be a busulfan or a hepsulfan. An aziridine may be a triethylenephosphoramide or a triethylenethiophosphoramide. An antineoplastic antibiotic may be selected from a group including, but not limited to, mitomycin A, mitomycin B, mitomycin C, amsacrine, actinomycin A, actinomycin C, actinomycin D, actinomycin F, carminomycin, daunomycin, 14-hydroxydaunomycin, mitoxantron, plicamycin and their analogs and derivatives. The nitrogen mustard, analogs or derivatives may be selected from a group including, but not limited to, chlorambucil, cyclophosphamide, ifosfamide, melphalan, mechloroethamine. The DNA reactive moiety can be a heavy metal coordination compound. The heavy metal coordination compound can be selected from a group including, but not limited to, carboplatin, cisplatin, transplatin, oxaliplatin and their derivatives and analogs.

Linkers that are Stable Under Intracellular Conditions

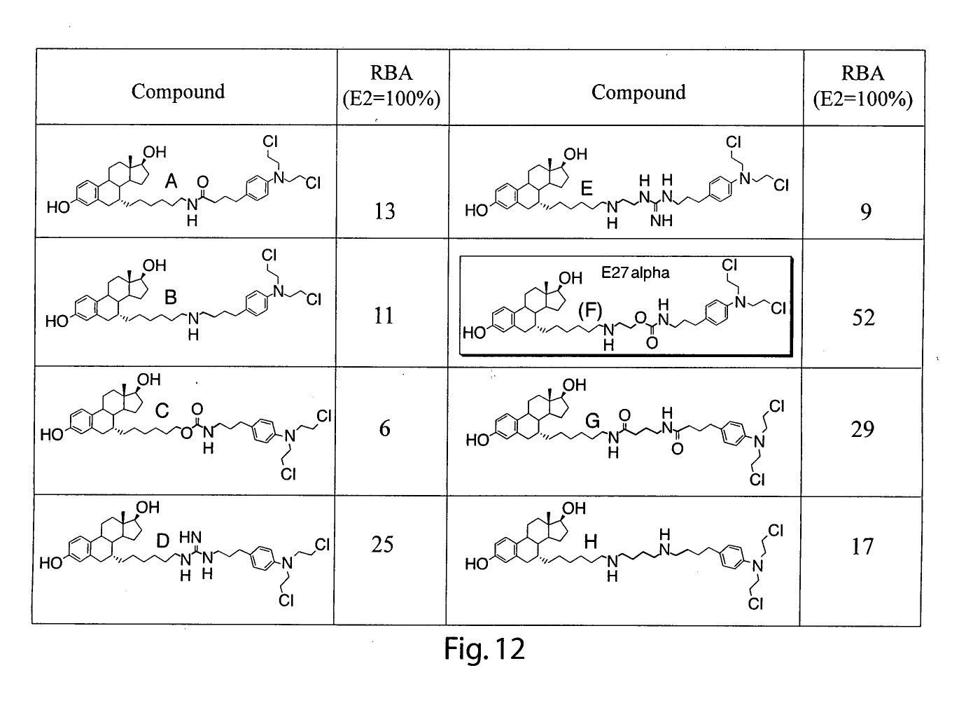

A compound of the invention comprises a linker that connect the ligand (e.g., protein recognition moiety) and the first moiety (e.g., the DNA alkylating moiety or variant thereof). According to aspects of the invention, suitable linkers may have one or more of the following properties: solubility under intracellular conditions, stability under intracellular conditions, and/or a length (e.g., a length of a carbon alkyl chain) that is therapeutically optimized (e.g., optimized to simultaneously allow compound-DNA interaction and compound-cellular protein interaction). In one embodiment, a linker may contain one or more polar or charged residues in order to improve solubility under intracellular conditions. In one embodiment, a linker may contain one or more carbamate(s) and/or one or more amine(s) (e.g., secondary amines) in order to increase solubility under intracellular conditions. Alternatively, or in addition, the linker may contain one or more sulfates. In certain embodiments of the invention, linkers may be alkyl-amino-carbamate alkyl chains of various lengths. In certain aspects of the invention linkers comprising amino, diamino, sulfate and carbamate groups are of particular importance. In one embodiment, a linker includes an alkyl chain that is 3-10 carbons in length. In certain preferred embodiments, the linker includes a six carbon alkyl chain. A linker may be attached (e.g., covalently) to any atom (e.g., any one or more of a C, N, S, O, or other atom) on the ligand and/or the first moiety. In certain embodiments, a polar or charged moiety (e.g., a carbamate, amine, sulfate or other polar or charged moiety) in the linker is preferably separated from the ligand (and/or first moiety) by one or more carbons (e.g., 2, 3, 4, 5, 6, etc.) so that the portion of the linker adjacent to the ligand (and/or the first moiety) is relatively non-polar or hydrophobic. This property may be useful to enhance ligand binding to a non-polar or hydrophobic molecule (e.g., certain steroid receptors). Linkers preferably do not contain bonds that are degraded or unstable under intracellular conditions. Accordingly, linkers preferably do not contain unstable or labile ureas, esters, or amides. FIG. 12 shows the relationship between compounds with different linkers and relative binding affinities (RBA) by cellular receptors.

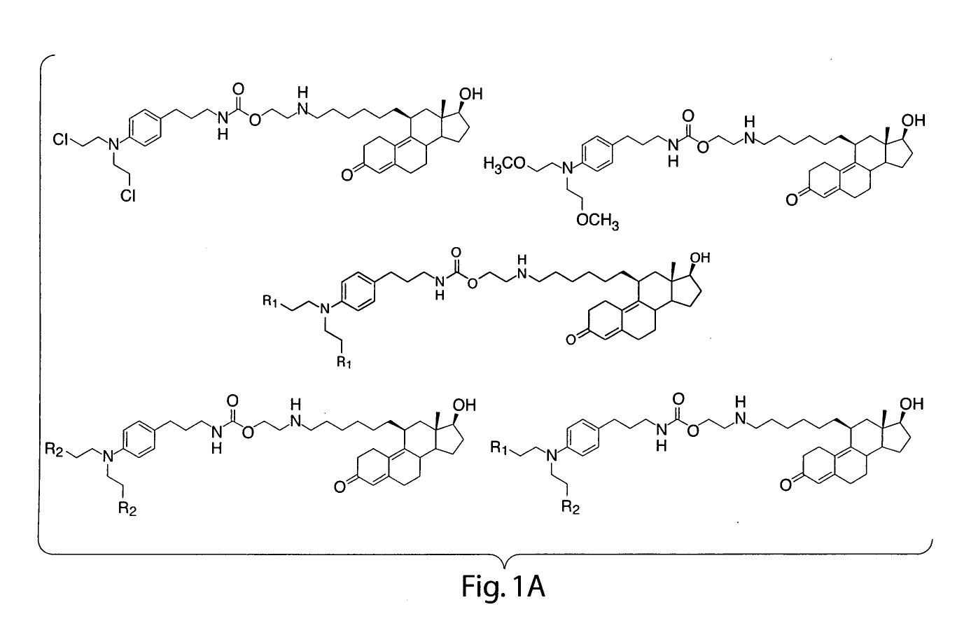

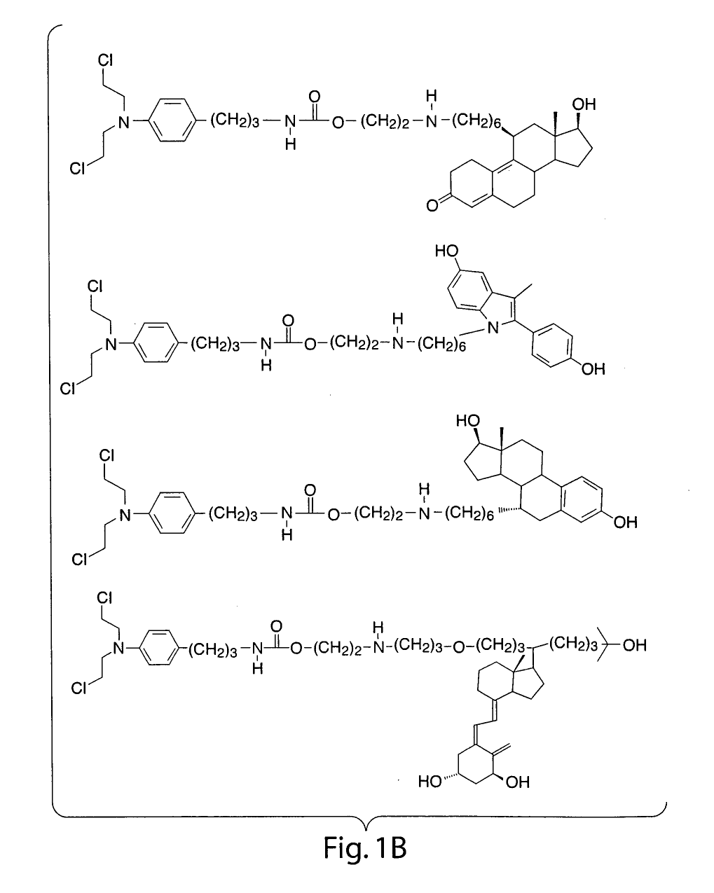

FIG. 1 shows non-limiting embodiments of compounds of the invention. In some embodiments, R₁ can be Cl or another good leaving group such as Br, I, or sulfonyl. In some embodiments, R₂ can be methoxy or other poor leaving group such as methyl or ethyl that will not form a reactive electrophile.

In one aspect, compounds of the invention are cytotoxic. In one embodiment, cytotoxic compounds have an alkylating nitrogen mustard domain (e.g. N,N-bis-2-chloroethylaniline). Examples of cytotoxic compounds are those that promote apoptosis. In another aspect, compounds of the invention are cytostatic. In one embodiment, cytostatic compounds have a non-alkylating moiety (e.g. N,N-bis-methoxyaniline or N,N-bis-3-propylaniline). Cytostatic compounds may be non-reactive analogs of alkylating compounds. Alternatively, cytostatic compounds may include analogs that are capable of forming a single covalent bond with a cellular target such as DNA (e.g., (N-2-cholorethyl)-(N-2-methoxyethyl)-aniline).

Compounds of the invention may have one or more of the following properties: alkylate DNA, interact with steroid receptors, interact with cellular proteins, interact with cellular components, induce apoptosis, induce cell cycle arrest, induce PARP cleavage, induce DNA fragmentation, increase p27 levels, increase p21 levels, decrease phosphorylation of p70S6K, decrease intracellular c-Myc levels, and/or decrease intracellular Skp2 levels. In some embodiments, cytotoxic compounds have all of the above properties. In some embodiments, cytostatic compounds induce cell cycle arrest, increase p27 levels, decrease phosphorylation of p70S6K, decrease c-Myc levels, and decrease Skp2 levels. In some embodiments, cytostatic and/or cytotoxic compounds also interact with steroid receptors (and/or other cellular proteins).

According to the invention, these properties confer useful anti-cancer activities on these compounds.

Accordingly, the invention also provides assays for testing the effectiveness of a compound for treating cancer. The assays can involve measuring or detecting any one or more of the following: DNA alkylation, apoptosis induction, cell cycle arrest, PARP cleavage, DNA fragmentation, increased p27 levels, increased p21 levels, decrease phosphorylation of p70S6K, decreased c-Myc levels, or decreased Skp2 levels. In addition, or instead, an assay may involve testing the cytotoxic and/or cytostatic effects of one or more compounds in an in vitro cell extract.

Synthesis Methods

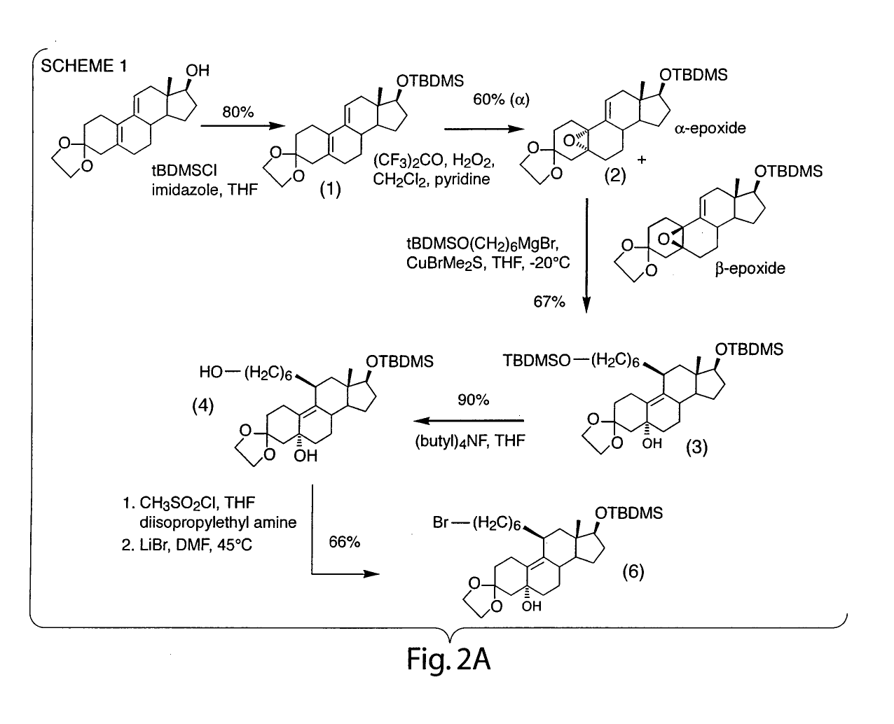

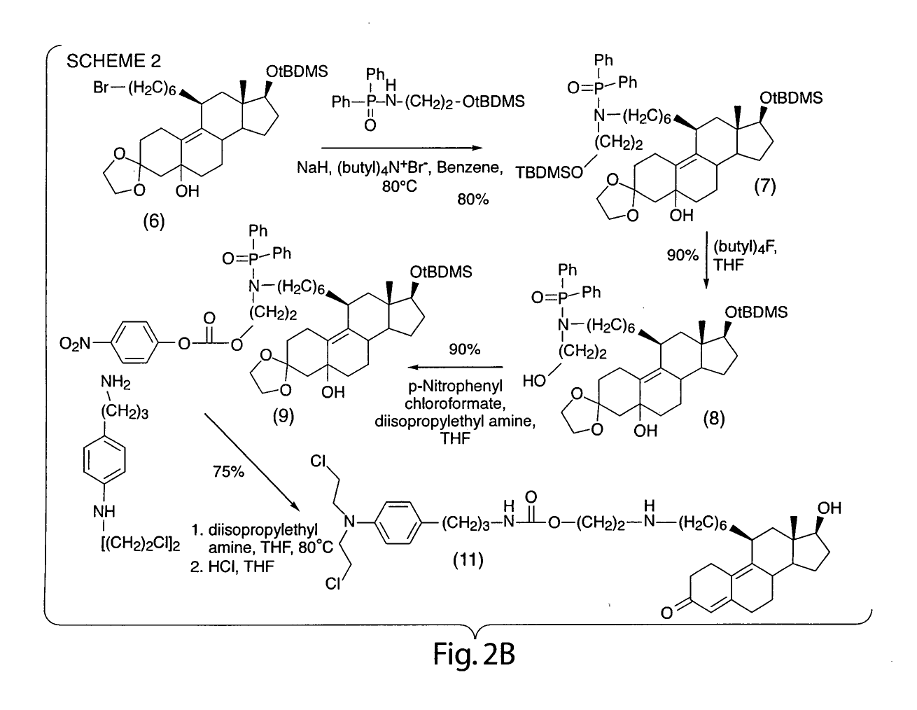

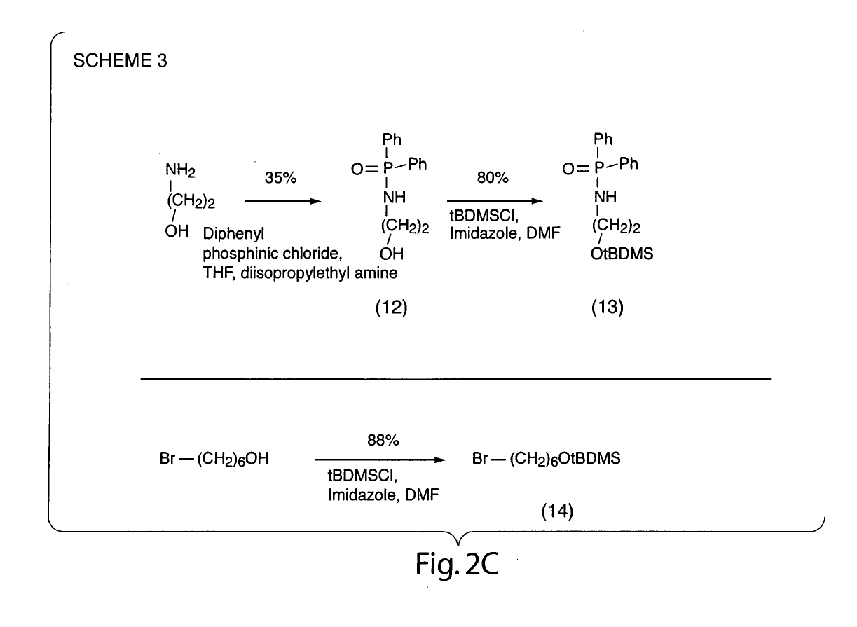

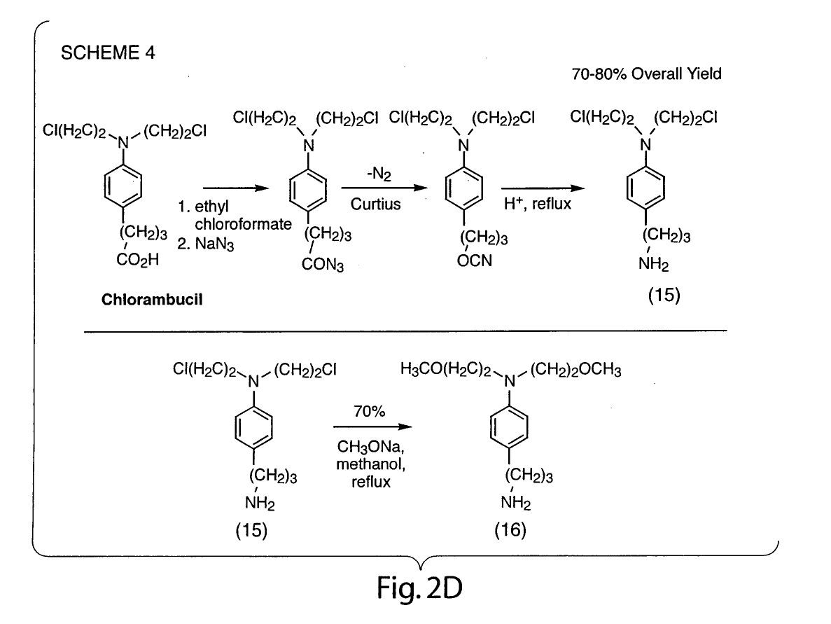

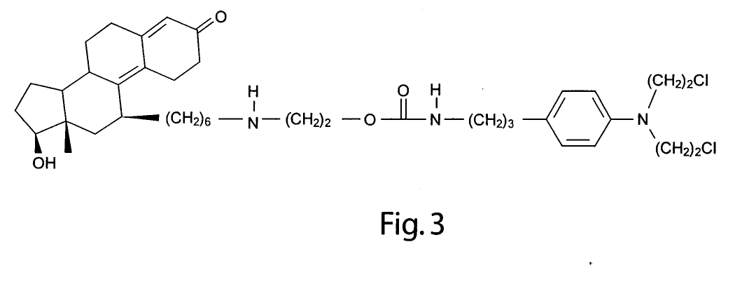

Aspects of the invention provide methods for synthesizing compounds useful for treating cancer. In general, compounds of the invention are synthesized using methods available in the scientific literature as well as those disclosed herein. FIG. 2A-D shows an embodiment of a synthetic method for preparing a dichloro derivative of the invention. Example 2 includes non-limiting examples of other synthetic methods of the invention. Aspects of the invention also provide modification to these synthetic methods that are useful for increasing efficiencies, reducing product cost, minimizing toxic side products, and/or producing modified compounds of the invention.

Applications

The invention provides methods for both in vitro and in vivo gene regulation. In some embodiments, compounds of the invention are useful for decreasing the expression or activities of one or more of the following genes: p70S6K, Skp2, p45 (or for decreasing the activity of the corresponding gene product). In some embodiments, compounds of the invention are useful for increasing the expression of one or more of the following genes: p27, p21 (or for increasing the activity of the corresponding gene product). In some embodiments, compounds of the invention are useful for killing cells, particularly cancer cells. In some embodiments, compounds of the invention are useful for stopping or slowing cell growth, particularly cancer cell growth. In some embodiments, compounds of the invention are useful for treating patients diagnosed with cancer or at risk of developing cancer.

According to aspects of the invention, methods of treating cancer include preventing, slowing the progression, curing, reducing the symptoms, and/or any other desired effect on cancer. Compounds of the invention can be administered prior to a cancer surgery, after a cancer surgery, or as part of any cancer therapeutic regimen including chemotherapeutic and radiotherapeutic treatments.

Aspects of the invention also provide methods for screening candidate compounds to identify useful anti-cancer agents. In some embodiments, a screen involves incubating one or more candidate compounds with a compound of the invention and assaying the combination in one of the assays of the invention. For example, a cytostatic compound of the invention can be added to a screen to identify compounds that are effective at killing growth-arrested cells. These screens can identify compounds that are useful alone or in combination therapies with other anti-cancer compounds. In particular, the invention provides methods for identifying compounds that are effective for treating (including killing) cells in G1 arrest. For example, cytostatic compounds of the invention can be used in screens to identify compounds that kill cells in G1 arrest.

In some embodiments, cytostatic compounds of the invention such as those containing dimethoxy groups can be used in combination with one or more other anticancer treatments. For example, such compounds can be administered along with a cisplatin-based therapeutic drug. This can be useful to reduce or minimize any side effects associated with one or more of the therapeutic agents.

In other embodiments, cytotoxic compounds such as the dichloro compounds of the invention can be used in combination with one or more other anticancer treatments. For example, such compounds can be administered along with a cisplatin-based therapeutic drug. When used in combination with other drugs, low doses of both the compounds of the invention and the additional anticancer drug can be used. This can be useful to reduce or minimize any side effects associated with one or more of the therapeutic agents (e.g. for assaying compounds or evaluating their potential effectivness to treat cancer).

Useful cells for certain methods of the invention include, but are not limited to, DLD-1 cells, Hela cells, and LNCaP cells.

Cancer Therapies

Methods and compounds of the invention are particularly useful for treating cancers that do not express certain steroid receptors. Compounds of the invention can be administered alone or in combination with one or more cancer drugs or therapies (including radiation, surgery, etc.).

Methods and compounds of the invention are also useful for treating cancers that have one or more of the following properties: they do not express steroid receptors, or they are resistant to conventional genotoxic therapeutics because of activation of pathways that inactivate apoptosis.

Accordingly, methods and compounds of the invention can also be useful to treat any cancer, including but not limited to: biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; esophageal cancer; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; transitional cancer and renal cancer including adenocarcinoma and Wilms tumor.

Accordingly, compounds of the invention can be administered to any multicellular subject to treat cancer. According to the invention, a subject is preferably a human subject. However, a patient can also be a mammalian patient including, but not limited to, a dog, cat, mouse, rat, goat, sheep, horse, cow, donkey, or pig. A subject is preferably a patient diagnosed with cancer. A patient can be diagnosed with cancer using any recognized diagnostic indicator including, but not limited to, physical symptoms, molecular markers, or imaging methods. A subject can also be a subject at risk of developing cancer (e.g. a subject that has been exposed to a carcinogen or other toxin, a subject with one or more genetic predispositions for cancer, a subject with symptoms of early cancer, or a subject that has been treated for cancer and is at risk of cancer recurrence or metastasis).

Other Diseases

Methods and compounds of the invention also may be useful to treat other diseases. As discussed above, one aspect of the invention provides methods for potentiating the effect of an alkylating agent. Accordingly, it is expected that compounds of the invention may be useful to treat one or more conditions that are currently treated with an alkylating drug. In one aspect, the invention provides methods for treating one or more of the following conditions using one or more alkylating compounds of the invention: psoriasis, autoimmune disorders such as multiple sclerosis, and/or inflammatory disorders that are susceptible to treatment with an alkylating agent.

Formulations

Compounds of the invention can be formulated in any appropriate manner for delivery to a cell such as a cell in culture or a cell in vivo. Accordingly, compounds of the invention can be formulated as therapeutic compositions for administration to a patient.

The present invention therefore provides pharmaceutical compositions comprising a one or more anti-cancer compounds or combinations thereof described herein. These pharmaceutical compositions may be administered orally, rectally, parenterally, intracistemally, intravaginally, intraperitoneally, topically (as by powders, ointments, drops or transdermal patch), bucally, or as an oral or nasal spray. As used herein, “pharmaceutically acceptable carrier” is intended to mean a non-toxic solid, semisolid or liquid filler, diluent, encapsulating material or formulation auxiliary of any type. The term “parenteral” as used herein refers to modes of administration which include, but are not limited to, intravenous, intramuscular, intraperitoneal, intrasternal, subcutaneous and intraarticular injection and infusion. One of ordinary skill will recognize that the choice of a particular mode of administration can be made empirically based upon considerations such as the particular disease state being treated; the type and degree of the response to be achieved; the specific agent or composition employed; the age, body weight, general health, sex and diet of the patient; the time of administration and rate of excretion of the agent or composition; the duration of the treatment; drugs (such as a chemotherapeutic agent) used in combination or coincidental with the specific composition; and like factors well known in the medical arts.

Pharmaceutical compositions of the present invention for parenteral injection may comprise pharmaceutically acceptable sterile aqueous or nonaqueous solutions, dispersions, suspensions or emulsions as well as sterile powders for reconstitution into sterile injectable solutions or dispersions just prior to use. Illustrative examples of suitable aqueous and nonaqueous carriers, diluents, solvents or vehicles include, but are not limited to, water, ethanol, polyols (such as glycerol, propylene glycol, polyethylene glycol, and the like), carboxymethylceuulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

The compositions of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the therapeutic agent or inhibitor, it is desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include, but are not limited to, capsules, tablets, pills, powders, and granules. In such solid dosage forms, the active compounds are preferably mixed with at least one pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents as appropriate.

Solid compositions of a similar type may also be employed as fillers in soft and hard filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Illustrative examples of embedding compositions which can be used include, but are not limited to, polymeric substances and waxes.

The compounds can also be in micro-encapsulated form, if appropriate, with one or more of the above-mentioned excipients.

Liquid dosage forms for oral administration include, but are not limited to, pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions may also contain adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The agent or inhibitor can also be administered in the form of liposomes. As is known to those skilled in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to the agent or inhibitor, stabilizers, preservatives, excipients, and the like. Preferred lipids are phospholipids and phosphatidyl cholines (lecithins), both natural and synthetic. Methods to form liposomes are known in the art. See, e.g., Prescott, ed., METHODS IN CELL BIOLOGY, Volume XIV, Academic Press, New York, N.Y. (1976), p. 33 et seq.

The compounds of the present invention can be formulated according to known methods to prepare pharmaceutically acceptable compositions, whereby these materials, or their functional derivatives, are combined in a mixture with a pharmaceutically acceptable carrier vehicle. Suitable vehicles and their formulation, inclusive of other human proteins, e.g., human serum albumin, are well known in the art. In order to form a pharmaceutically acceptable composition suitable for effective administration, such compositions will contain an effective amount of one or more compounds of the present invention.

Additional pharmaceutical methods may be employed to control the duration of action. Controlled release preparations may be achieved through the use of polymers to complex or absorb the therapeutic agents of the invention. The controlled delivery may be exercised by selecting appropriate macromolecules (such as polyesters, polyamino acids, polyvinyl, pyrrolidone, ethylenevinylacetate, methylcellulose, carboxymethylcellulose, or protamine sulfate) and methods of incorporation in order to control release. Another possible method to control the duration of action by controlled release preparations is to incorporate antibodies into particles of a polymeric material such as polyesters, polyamino acids, hydrogels, poly(lactic acid) or ethylene vinyl acetate copolymers. Alternatively, instead of incorporating these agents into polymeric particles, it is possible to entrap these materials in microcapsules prepared, for example, by coacervation techniques or by interfacial polymerization, for example, hydroxymethylcellulose or gelatine-microcapsules and poly(methylmethacylate) microcapsules, respectively, or in colloidal drug delivery systems, for example, liposomes, albumin microspheres, microemulsions, nanoparticles, and nanocapsules or in macroemulsions.

The pharmaceutical formulations of the present invention are prepared, for example, by admixing the active agent with solvents and/or carriers, optionally using emulsifiers and/or dispersants, whilst if water is used as the diluent, organic solvents may be used as solubilizing agents or auxiliary solvents. As described above, the excipients used include, for example, water, pharmaceutically acceptable organic solvents such as paraffins, vegetable oils, mono- or polyfunctional alcohols, carriers such as natural mineral powders, synthetic mineral powders, sugars, emulsifiers and lubricants.

One of ordinary skill will appreciate that effective amounts of the therapeutic compounds can be determined empirically and may be employed in pure form or, where such forms exist, in pharmaceutically acceptable salt, ester or prodrug form. The compound can be administered in compositions in combination with one or more pharmaceutically acceptable excipients. It will be understood that, when administered to a human patient, the total daily usage of the agents and compositions of the present invention will be decided by the attending physician within the scope of sound medical judgment. The specific therapeutically effective dose level for any particular patient will depend upon a variety of factors including the type and degree of the response to be achieved; the specific agent or composition employed; the age, body weight, general health, sex and diet of the patient; the time of administration, route of administration, and rate of excretion of the agent or composition; the duration of the treatment; drugs (such as a chemotherapeutic agent) used in combination or coincidental with the specific composition; and like factors well known in the medical arts.

Techniques of dosage determination are well known in the art. The therapeutically effective dose can be lowered if a compound of the present invention is additionally administered with another compound. As used herein, one compound is said to be additionally administered with a second compound when the administration of the two compounds is in such proximity of time that both compounds can be detected at the same time in the patient's serum.

For example, satisfactory results are obtained by oral administration of therapeutic dosages on the order of from 0.05 to 10 mg/kg/day, preferably 0.1 to 7.5 mg/kg/day, more preferably 0.1 to 2 mg/kg/day, administered once or, in divided doses, 2 to 4 times per day. On administration parenterally, for example by i.v. drip or infusion, dosages on the order of from 0.01 to 5 mg/kg/day, preferably 0.05 to 1.0 mg/kg/day and more preferably 0.1 to 1.0 mg/kg/day can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v.

Dosaging may also be arranged in a patient specific manner to provide a predetermined concentration of a compound in the blood, as determined by the RIA technique. Thus patient dosaging may be adjusted to achieve regular on-going trough blood levels, as measured by RIA, on the order of from 50 to 1000 ng/ml, preferably 150 to 500 ng/ml. In some embodiments, compounds of the invention are provided at a concentration of between 1 μM and 1 mM, and preferably at about 5-10 μM. However, the compounds may be provided at lower or higher concentrations.

Pharmaceutical compositions of the invention may also include one or more targeting agents to direct an anti-cancer compound to a specific cancer type or tissue type. Alternatively or additionally, pharmaceutical preparations of the invention can be injected or otherwise administered into or near a cancer or tumor in a patient. However, in some embodiments a systemic administration may be preferred, either to treat a systemic cancer or to minimize the risk of metastasis.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

### EXAMPLES

### Example 1

### Multifunctional Compounds

FIG. 1 shows embodiments of compounds of the invention. The key molecular feature of these compounds that is central to their biological activity and efficacy is the linker that connects the steroid and aniline moieties. The linker is constructed such that it maintains the biophysical and biological properties of the pharmacophores at either end.

### Example 2

### Compound Synthesis

Synthesis of 11β Compounds Starting from the Known Compound 17β-OH-(3,3-ethylenedioxy-estra-5(10),9(11)-diene).

Scheme 1:

Silylation of the starting compound; 17β-OH-(3,3-ethylenedioxy-estra-5(10),9(11)-diene). To 2.2 gm (6.98 mmol) of the starting compound 17β-OH-(3,3-ethylenedioxy-estra-5(10),9(11)-diene) dissolved in 30 DMF was added imidazole (1.42 gm, 21 mmol) and tBDMSiCl (2.11, 14 mmol). After 3 h the reaction was complete as monitored by TLC. 200 ml of H₂O was added and the cloudy suspension was extracted with EtOAc (200 ml) and then twice with additional 50 ml EtOAc. The combined organic fractions were extracted once with H2O and dried over Na₂SO₄. After removing the solvent under reduced pressure, the product (1) was purified by flash chromatography on silica gel (20:1, hexanes:EtOAc) to yield a white solid (1.8 gm, 80%)

Epoxidation of (1) To 2.8 gm (4 mmol) of (1) dissolved in 25 ml of CH₂Cl₂ was added 0.45 ml of (CF₃)₂CO and 0.45 ml of pyridine. After cooling to 0° C., 2 ml of 30% H₂O₂ was added dropwise. The reaction was allowed to warm to room temperature overnight. Water (100 ml) and CH₂Cl₂ were added and the organic phase separated and washed with Na₂S₂O₃ (sat.), brine solution and H₂O. The organic phase was dried over Na₂SO₄, and the products isolated by chromatography on alumina gel (Al₂O₃, Activity II-III) (hexanes:EtOAc 5 to 10% gradient). The ∀-epoxide (2) (1.32 gm, 46%) was isolated as a white foamy solid.

Preparation of the Grignard and alkylation of (2): 3.95 gm (13.4 mmol) of (6-bromohexyloxy)-tert-butyl-dimethyl-silane was dissolved in 4 ml of THF and added to 0.31 gm (12.8 mmol) of Mg. The reaction was warmed to 60° C. for 4 hr. After the Grignard was formed 8 ml of THF was added and the solution cooled to −20° C. Copper(I)bromide-dimethylsulfide complex (0.28 gm, 1.34 mmol) was added to the stirred suspension followed by 1.05 gm (2.35 mmol) of the α-epoxide (2) dissolved in 8 ml THF. The reaction was maintained at −20° C. for 1.5 h. The reaction mixture was then added to a flask containing 100 ml of EtOAc and 100 ml of NH₄Cl (sat) solution and stirred for 10 min. The organic phase was separated and the aqueous phase extracted twice with 50 ml of EtOAc. The combined organic fractions were washed once with H₂O and dried over K₂CO₃. The product (3) was isolated by chromatography on alumina (hexanes:EtOAc, 9:1) as a colorless oil (1.02 gm, 64%).

Removal of the silyl group from the 1° alcohol of (3). 1.8 gm (2.7 mmol) of (3) was dissolved in 20 ml of THF and 4.6 ml of a 1 M solution of (butyl)₄NF in THF was added and the reaction stirred at room temperature for 3 h. THF was removed by evaporation under vacuum leaving a syrup. The crude mixture was dissolved in hexanes/EtOAc/CH₂Cl₂ (1:3:1) and filtered through a short column of alumina producing 1.7 gm of a viscous syrup that was used without further purification.

Preparation of the methylsulfonic acid ester of (4). 1.5 gm (2.7 mmol) of the alcohol (4) was dissolved in 25 ml of THF and 1.4 ml (8 mmol) of diisopropylethyl amine and the solution cooled to 0° C. A solution of 0.3 ml (3.9 mmol) of MeSO₂Cl in 10 ml THF was added. After 1 h 100 ml of NaHCO₃ solution (sat) and the mixture extracted with 100 ml EtOAc. The organic phase was separated and the aqueous phase extracted twice with 50 ml EtOAc. The combined organic fractions were washed with H₂O and dried over K₂CO₃, producing 1.7 gm of (5) as a yellowish oil. (Structure of 5 is not shown in synthetic scheme.) The product was used without further purification.

Bromo substitution of the methylsulfonic acid ester (5). To a stirred solution of 1.7 gm (2.7 mmol) of the methylsulfonate (5) dissolved in 20 ml DMF was added 0.7 gm (8 mmol) LiBr and the solution heated to 45° C. for 3 h. The reaction was cooled and 100 ml of NaHCO₃ solution (sat) was added followed by 100 ml EtOAc. The organic phase was separated and the aqueous phase extracted twice with 50 ml EtOAc. The combined organic fractions were washed with H₂O and dried over K₂CO₃. The product (6) was isolated by chromatography on alumina (9:1, hexanes:EtOAc) producing 1.1 gm of a colorless oil that became a white solid on standing.

Scheme 2:

Reaction of the alkylbromide (6) with 2-(tert-butyldimethylsilanoxy)ethyl-diphenylphosphinamide: The alkylbromide (2.3 gm, 3.8 mmol) was dissolved in 20 ml benzene and 2.9 gm (7.7 mmol) of the phosphinamide, 0.25 gm (0.77 mmol) of tetrabutylammonium bromide and 0.24 gm (10 mmol) of NaH were added to the solution that was maintained under Argon gas. The solution was heated to 65° C. for 3.5 h. After cooling, 100 ml of NaHCO₃ solution (sat) was added and the benzene phase separated and washed once with H₂O. The combined aqueous phases were then extracted three times with 50 ml CH₂Cl₂. The combined organic phases were dried over K₂CO₃ and solvents removed under vacuum. The crude product (7) (3.46 gm) was used in the next reaction without further purification.

Removal of the tert-butyldimethylsilanoxy group from the 1° alcohol of (7). 3.46 gm of crude product from the last step was dissolved in 25 ml of benzene and 3.9 ml (3.9 mmol) of a 1 M solution of tetrabutlyammonium fluoride as added. After 3 h the solvent was evaporated under reduced pressure. The crude product was dissolved in CH₂Cl₂ and isolated by chromatography on alumina (Al₂CO₃, activity II-III) (CH₂Cl₂:MeOH, 98:2) producing 3 gm of (8) as a white solid.

Synthesis of the p-nitrophenyl carbonate (9). 3 gm (3.75 mmol) of the 1o alcohol (8) obtained from the previous step was dissolved in 50 ml of THF containing 2.3 ml (13.2 mmol) of diisopropylethyl amine. To this solution was added 1.5 gm (7.44 mmol) of p-nitrophenylchloroformate dissolved in 10 ml THF. After 3 h, 250 ml of Na₂CO₃ (sat) solution was added to the reaction followed by 300 ml EtOAc. The organic phase was separated and extracted 4× with 100 ml Na₂CO₃ solution (sat) followed by 200 ml H₂O. The organic phase was dried over Na₂SO₄ and solvents removed under reduced pressure yielding 4.6 gm of crude product (9). This material was used in the next reaction without further purification.

Reaction of 4-(N,N-bis-2-chloroethylaminophenyl)-propylamine with p-nitrophenyl carbonate (9). 4 gm of crude product (estimate=3.1 mmol) from the previous reaction were dissolved in 20 ml of THF containing 2.1 ml (12 mmol) of diisopropylethyl amine. To this solution was added 1.45 gm (5.3 mmol) of 4-(N,N-bis-2-chloroethylaminophenyl)-propylamine dissolved in 8 ml THF. The reaction was heated at 75-80° C. for 3 h after which the THF was removed under reduced pressure producing a yellow syrup which was dissolved in 200 ml EtOAc. The EtOAc solution was extracted 4× with 100 ml Na₂CO₃ solution (sat), 2× with H₂O and then dried over Na₂SO₄. The product (10) was purified by flash chromatography on silica gel eluted stepwise [(1) CH₂Cl₂; (2) CH₂Cl₂:EtOAc:MeOH (55:44.5:0.5); (3) CH₂Cl₂:EtOAc:MeOH (55:43:2)] yielding 2.5 gm of white solid. This represents an overall yield of 74% for the last 4 steps beginning with the bromide (Step 7).

Removal of tert-butyldimethylsilanoxy- and phosphinamido-groups from compound (10). 2.5 gm of compound (10) were dissolved in 50 ml of THF and 2 ml of HCl (conc) were added dropwise. After 5 h the reaction was neutralized by addition of solid NaHCO₃. The resulting suspension was filtered through Celite and the solvent removed under reduced pressure. The product (11) was purified by flash chromatography on silica gel eluted stepwise [(1) CH₂Cl₂:EtOAc:MeOH (3.5:6:0.5); (2) CH₂Cl₂:EtOAc:MeOH (3:6:0.7); (3) CH₂Cl₂:EtOAc:MeOH (3:6:1)] producing 1.2 gm of white solid.

Scheme 3:

Preparation of 2-(hydroxy)ethyl-diphenylphosphinamide. (12) 5 ml (82.8 mmol) of ethanolamine was dissolved in 80 ml of DMF containing 15 ml (86.1 mmol) of diisopropylethylamine. After cooling the solution to 0° C. under argon, 10 gm (42.2 mmol) of diphenylphosphinic chloride was added. After 3 hr, 300 ml of H₂O and 300 ml of EtOAc were added and the organic phase separated and dried over Na₂SO₄. The product (12) was isolated by flash chromatography on silica gel CH₂Cl₂:MeOH (9:1) producing 3.8 gm a thick syrup (35% yield).

Preparation of 2-(tert-butyldimethylsilanoxy)ethyl-diphenylphosphinamide. (13) 3.8 gm (14.5 mmol) of 2-(hydroxy)ethyl-diphenylphosphinamide (12) was dissolved in 30 ml of DMF along with 2.5 gm (36 mmol) of imidazole. To this solution was added 2.41 gm (15 mmol) of tBDMSiCl. After 3 h, 200 ml of H₂O and 200 ml of EtOAc were added and the organic phase separated and dried over Na₂SO₄. The solvent was removed under reduced pressure yielding 4.6 gm of a thick syrup that became a white solid under vacuum overnight. The product (13) was subsequently recrystallized from hexanes (80% yield).

Preparation of (6-bromo-hexyloxy)-tert-butyl-dimethyl-silane. (14) 13.7 gm (75.4 mmol) of 6-bromo-1-hexanol was dissolved in 100 ml DMF to which was added 15.4 gm (226 mmol) imidazole. The solution was cooled to 0° C. and 20 gm (133 mmol) tert-butyl-dimethyl-silyl-chloride was added. After 1 h the solution was added to 500 ml H₂O. The cloudy suspension extracted with 200 ml EtOAc and again with 150 ml EtOAc. The combined organic phases were dried over Na₂SO₄ and the solvent evaporated under reduced pressure to produce a clear oil. The product (14) was purified by flash chromatography on silica gel eluted with hexanes:EtOAc (99:1); 19.3 gm (88%).

Scheme 4:

Preparation of 4-(N,N-bis-2-chloroethylaminophenyl)-propylamine from chlorambucil. (15) 5 gm (16.4 mmol) of chlorambucil was dissolved in 15 ml acetone and cooled to 0° C. The solution was treated with 2.5 ml Et₃N (17.9 mmol) and 1.7 ml ethylchloroformate (17.8 mmol). After 10 min, 2.14 gm NaN₃ (32.9 mmol) dissolved in 10 ml of H₂O was added and the mixture stirred for 30 min. The mixture was then poured into 300 ml of ice-cold H₂O and extracted 2× with 150 ml of toluene. The combined organic fractions were dried over MgSO4, filtered and heated under reflux for 1.5 h The solvents were removed and the residue dissolved in 25 ml of 8N HCl, which was heated under reflux for 10 min. The cooled mixture was then diluted with NaHCO₂ solution (sat) until neutralized and extracted 2× with 200 ml CH₂Cl₂. The combined organic phases were washed with 200 ml brine and dried with Na₂SO₄. Removal of solvents under reduced pressure produced 4 gm (88% yield) of (15) as a brownish syrup. For storage, the HCl salt was prepared by dissolving the product in CH₂Cl₂ and adding HCl (conc). The solvents were then removed under reduced pressure and the product recrystallization from EtOAc/MeOH.

Preparation of 4-(N,N-bis-2-methoxyethylaminophenyl)-propylamine (16). 1.0 gm (3.2 mmol) of 4-(N,N-bis-2-chloroethylaminophenyl)-propylamine (15) was dissolved in 83 ml of MeOH and 45 ml of a 0.5 M solution of CH₃ONa (22.46 mmol) was added. The solution was heated to reflux overnight. The solution was cooled, 250 ml of H₂O added and the aqueous phase extracted with 200 ml EtOAc. The EtOAc phase was washed with brine solution dried over Na₂SO₄ and the solvent removed under reduced pressure. The product (16) was purified by flash chromatography on silica gel eluted with CH₂Cl₂:MeOH:Et₃N (93:5:2); 0.6 gm (70%).

### Example 3

### Experiments

Evaluation of Prostate Anticancer Agents That Target Multiple Biochemical Pathways

Descriptions of the synthetic procedures for the 11β-dichloro and 11β-dimethoxy compounds are shown in FIG. 2. The compounds in FIG. 1 contain an 11β-substituted estradien-3-one, a pharmacophore that can bind to both the androgen and progesterone receptors. The linker that connects the steroid and the aniline mustard was designed to be stable to degradation by proteases and esterases, so that DNA adducts in vivo would be formed by the intact molecule. HPLC data on intact compound in mouse blood in vivo and data on DNA adduct formation in liver show conclusively that this linker is biologically stable. Competitive binding studies using the AR from LNCaP cells revealed that 11β has an RBA (Relative Binding Affinity) of 33 compared to the natural ligand dihydrotestosterone (i.e., the RBA of 11β was 33% that of dihydrotestoterone, for which RBA=100).

AR positive LNCaP cells were 7-fold more sensitive than AR negative PC3 and DU145 cells at a dose of 10 μM. Investigations on similar molecules in which a ligand for the estrogen receptor (ER) was tethered to the same DNA damaging warhead, a similar (but slightly smaller) differential toxicity in favor of killing ER positive cells was observed.

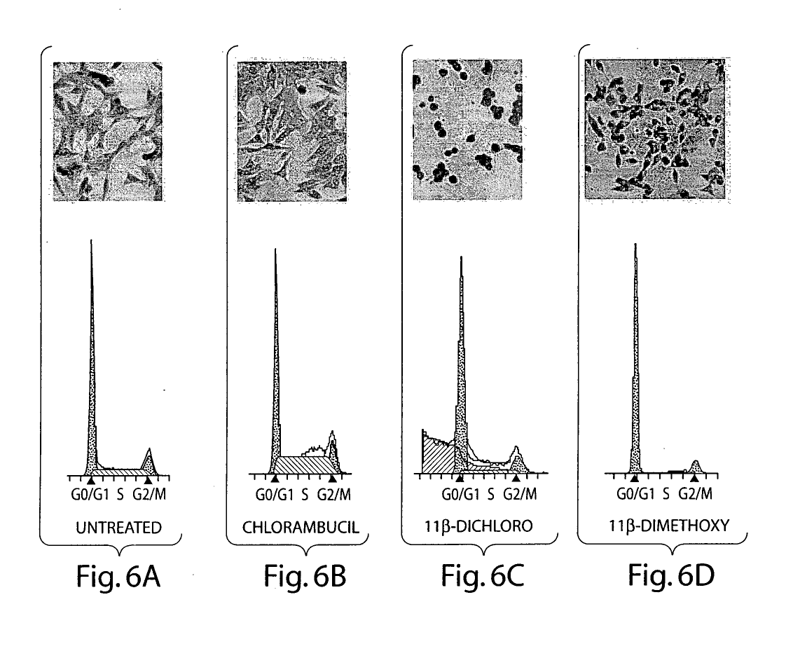

In cell culture, the cytotoxic effects of 11β-dichloro were compared with those of Chlorambucil, a clinically used nitrogen mustard antitumor drug that is expected to create DNA lesions similar to those of 11β-dichloro (purine monoadducts and inter and intrastrand crosslinks). Chlorambucil, unlike 11β-dichloro, lacks the linker and a ligand for the AR. Initial observations indicated a striking difference in the responses of LNCaP cells to these two compounds. Changes in the shape of LNCaP cells after 6 hr of exposure to 20 μM Chlorambucil, 10 μM 11β-dichloro or 10 μM 11β-dimethoxy were observed. FIG. 6 shows images of LNCaP cell morphology and cell cycle analysis of LNCaP cells treated with 11β compounds. Top: LNCaP cells after 6 h treatment with 11β compounds (10 μM) or the anticancer drug chlorambucil (20 μM). Cells in exponential growth phase were treated for 6 h, fixed, and stained with Giemsa. (A) Vehicle-treated LNCaP cells. (B) Cells exposed to chlorambucil showed no effect on cellular shape. (C) Cells treated with 11β-dichloro showed dramatic contraction and detachment. (D) Cells treated with the unreactive 11β-dimethoxy showed slight contraction, which was reversed by 24 h (not shown). Bottom: Cell cycle analysis of LNCaP cells treated with indicated compounds for 17 h.

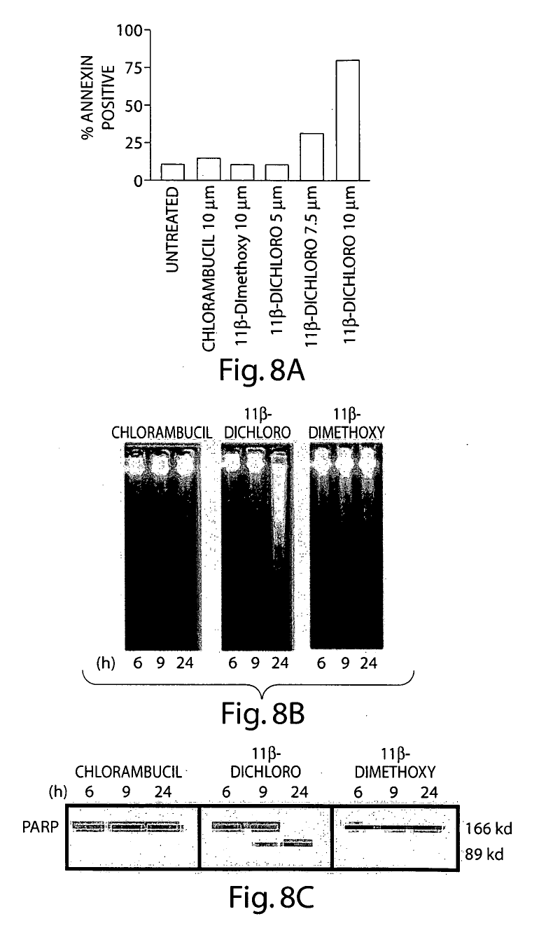

Cells treated with Chlorambucil remain spread out and appear unaffected while the 11β-treated cells are rounded and have undergone cytoplasmic contraction. The morphological changes induced by 11β-dichloro suggested activation of an apoptotic response. This suspicion was confirmed by analysis of PARP and Bid cleavage, and DNA fragmentation. FIG. 8 shows that LNCaP cells undergo apoptosis upon yreatment with 11β-dichloro: (A) Annexin V staining of LNCaP cells after treatment for 15 h with indicated compounds. Cells treated with >5 μM 11β-dichloro showed evidence of increased Annexin V staining. (B) Agarose gel electrophoresis of DNA isolated from LNCaP cells after 24 h exposure to chlorambucil (20 μM), 11β-dichloro (10 μM) or 11β-dimethoxy (10 μM). DNA fragmentation occurs in cells treated with 11β-dichloro. (C) Western blot analysis of cellular extracts probed with antibodies against full length and cleaved PARP showed that treatment with 11β-dichloro (10 μM) led to cleavage of PARP within 9 h. No cleavage was seen in cells treated with either chlorambucil (20 μM) or 11β-dimethoxy (10 μM). No changes in PARP, Bid nor DNA fragmentation were evident in Chlorambucil-treated cells. In pharmacokinetic studies done in mice, plasma concentrations of 10 to 40 μM C-11β-dichloro were achieved for more than two hours after injection of a dose of 10 μM 11β-dichloro, a dose that causes LNCaP tumor inhibition when given chronically (animal studies are described below). Therefore the doses used in these cell culture experiments aimed at detection of apoptosis are realistic ones.

The synthesis of an unreactive dimethoxy analog of 11β-dichloro in which the two chlorine atoms of the N,N-bis(2-chloroethyl) aniline moiety were replaced by methoxy groups allowed the cytotoxic mechanism(s) of 11β-dichloro to be probed. The chemical modification resulting in 11βdimethoxy maintained the physical chemical properties of the original molecule while eliminating its ability to form a reactive aziridinium ion that alkylates DNA. The 11β-dimethoxy molecule did induce changes in LNCaP cell shape, but these changes were reversible and were less dramatic than those observed with the 11β-dichloro compound. The most interesting effect of the “inactive” compound, however, was its ability to halt, albeit transiently, the growth of LNCaP cells in the G1 phase of the cell cycle. Flow cytometry revealed >90% of LNCaP cells in G1 after exposure to 10 uM 11β-dimethoxy for 20 hr. The dimethoxy compound did not activate an apoptotic response—indicating that a chemically reactive form of the molecule is required for this effect. The 11β-dichloro compound, in contrast to the dimethoxy analog, did not arrest cells in a specific point in the cell cycle but was a potent inducer of apoptosis and cell death. Taken together, these results began to unveil the fact that 11β-dichloro, which was designed to have two mechanisms of action, may have additional unanticipated mechanism(s) by which it kills cells.

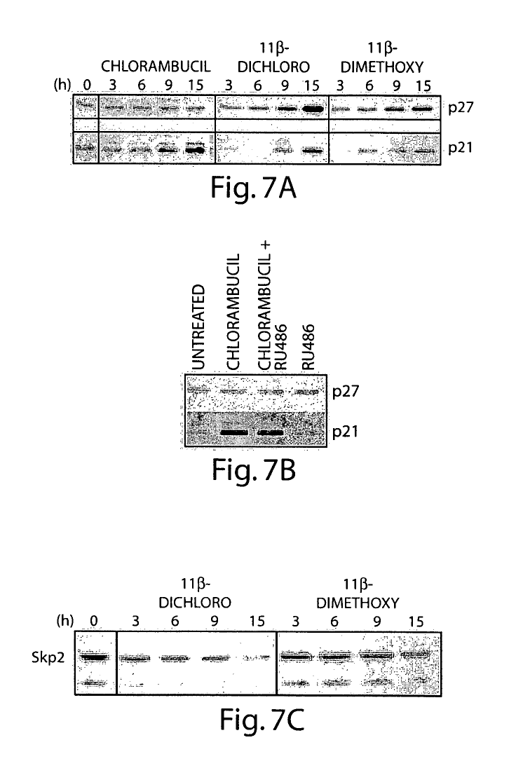

The ability of the dimethoxy compound to cause G1 arrest led us to investigate the effects of both 11β compounds (the DNA-reactive molecule and the one that had its warhead inactivated) on the expression of the G1 checkpoint CDK inhibitor proteins p27 (Kip1) and p21 (WAF1/Cip1) as shown in FIG. 7: (A) Levels of p21^{CIP1}, p27^{KIP1} in extracts from LNCaP cells that were treated with chlorambucil (20 μM), 11β-dichloro (10 μM), or 11β-dimethoxy (10 μM) for up to 15 h. (B) Levels of p21^{CIP1} and p27^{KIP1} in extracts of LNCaP cells treated for 15 h with chlorambucil (10 μM), RU486 (10 μM) or both (each at 10 μM). (C) Levels of Skp2 in extracts of LNCaP cells treated under the same conditions as in (A).

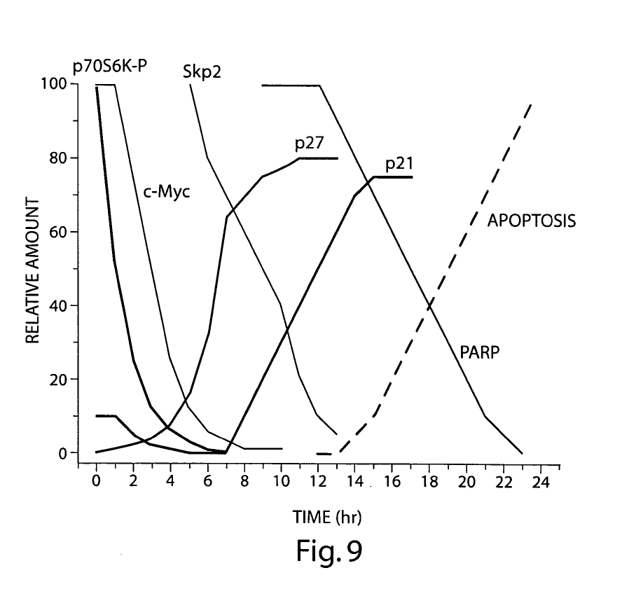

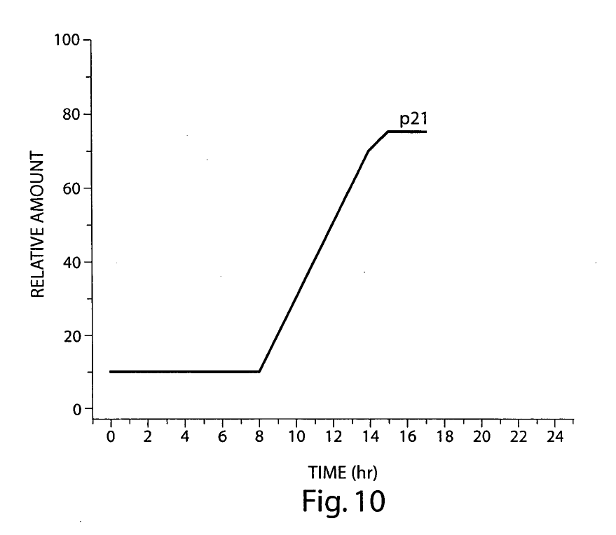

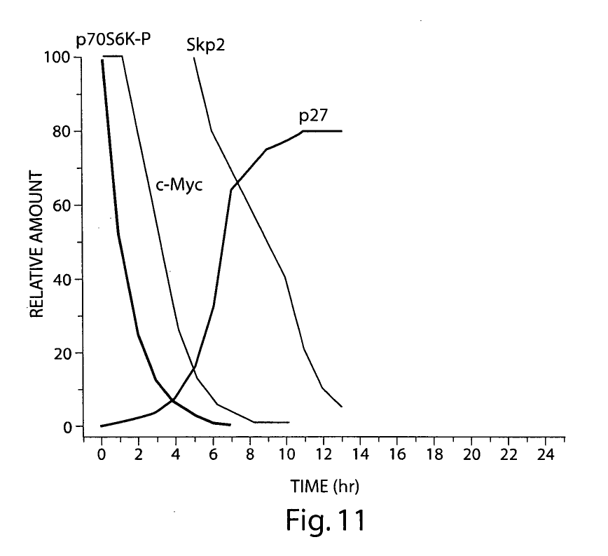

The parameters observed are summarized in cartoon format in FIGS. 9-11. Western analyses revealed that levels of p27 were increased by both the 11β-dichloro and 11β-dimethoxy compounds whereas Chlorambucil was without effect. In contrast, Chlorambucil was found to be a potent inducer of p21, and only p21. Levels of p21 initially decreased in cells treated with either the 11β-dimethoxy or 11βdichloro compound. Eventually the levels of p21 recovered to basal levels in 11β-dimethoxy-treated cells and remained stable. The level of p21 increased 9-fold in cells treated with 11β-dichloro. Thus, there is a markedly different pattern of activation of G1 checkpoint proteins in LNCaP cells treated with the 11β compounds as compared to Chorambucil.

Further pursuit of the pathways responsible for increased expression of p27 led us to examine levels of p45 Skp2, the F-box component of an SCF ubiquitin ligase complex that regulates degradation of p27. Once again we found a remarkable difference in the responses of LNCaP cells treated with either Chlorambucil or the two 11β compounds. Levels of Skp2 decreased in cells treated with 11βdichloro or -dimethoxy but were unaffected by Chlorambucil.

Two other biochemical changes were identified in LNCaP cells treated with the 11β compounds that are absent in cells treated with other alkylating compounds such as Chlorambucil. Levels of the c-Myc protein decreased rapidly following addition of 11β-dichloro. This result led us to investigate of the status of the p70S6K protein, which in some cells can lie upstream of c-Myc in its regulatory network. Since it was reported that inhibition of the mTOR kinase by rapamycin strongly inhibited translation of c-Myc, we decided to examine a target of mTOR, namely p70S6K. Strikingly, we found that the phosphate on Th389 of p70S6K was removed within 30 min of addition of 11β-dichloro to cells. Examination of another protein target of mTOR, 4E-BP1 also revealed the rapid disappearance of phosphate groups that would lead to its activation and inhibition of the initiation factor e1F4E.

The temporal series of molecular changes in LNCaP cells treated with 11β-dichloro are summarized in FIG. 9. These changes clearly distinguish the mechanism(s) of action of this new compound from several of the alkylating drugs that are in clinical use. Among the biochemical changes shown in FIG. 9 only one—the induction of p21—was observed in LNCaP cells after treatment with Chlorambucil. The ability of the 11β-dichloro compound to activate apoptosis efficiently in LNCaP cells may be particularly important for its therapeutic potential. The uniqueness of this compound is underscored by an experiment in which LNCaP cells were treated with a combination of Chlorambucil and 11β-dimethoxy. This combination did not induce apoptosis indicating that the unique responses to the 11β-dichloro compound are not simply a combination of those independently produced by the AR interactive ligand and the reactive N,N-bis(2-chloroethyl) aniline. Additionally, we found that Chlorambucil given along with the antiprogestin, RU486, which is also an AR antagonist, did not induce p27 nor result in apoptosis. This result again emphasizes that it is critical to have the steroid and DNA damaging moieties linked in order to achieve the biological effects observed with 11β-dichloro. According to the invention, certain compounds are capable of inhibiting a key component of the mTOR pathway as well as acting as a genotoxin by forming DNA adducts. Effects of 11β-dichloro on the mTOR pathway precede apoptosis and may play a role in events responsible for cell death.

Accordingly, the biochemical effects of Chlorambucil, 11β-dichloro, and 11β-dimethoxy in LNCaP cells are contrasted as follows:
1. Chlorambucil induces p21, but none of the other changes in FIG. 9, and fails to induce apoptosis.
2. 11β-dimethoxy inhibits p70S6K, causes c-Myc levels to drop, and later is associated with a decrease in Skp2 activity. Finally, its administration results in p27 increase and G1 arrest. The compound has a transient toxic effect but does not kill cells. It does not induce markers of apoptosis.
3. 11β-dichloro causes all of the above changes and, in addition, activates an apoptotic response resulting in destruction of cells.

In Vivo Evaluation of the Antitumor and Other Biological Properties of 11β-dichloro.

A set of in vivo studies was performed to assess the stability and biodistribution of the 11β-dichloro compound in mice. These studies required formulation of the compound in a vehicle that would deliver the compound to the tissues following IP or IV administration. The vehicle was Cremophor EL:ethanol:saline (40:30:30 by volume). Administration of radiolabeled [¹⁴C]-11β-dichloro via IP injection resulted in rapid compound absorption with plasma levels reaching the 30-40 μM range within 30 min. HPLC analyses found lower concentrations of the intact compound in plasma at 1 hour along with several minor unidentified metabolites. For 2 to 3 hours the compound was present above the 10 mM concentration at which good differential toxicity in favor of killing LNCaP cells in culture was observed. There was also evidence that the intact molecule reached its intended biological target—cellular DNA. 11β-dichloro-DNA adducts formed by the intact compound were isolated from liver DNA of treated animals two hours post dosing. The presence of these adducts indicates that 11β-dichloro has sufficient stability to penetrate tissues and react with cellular DNA.

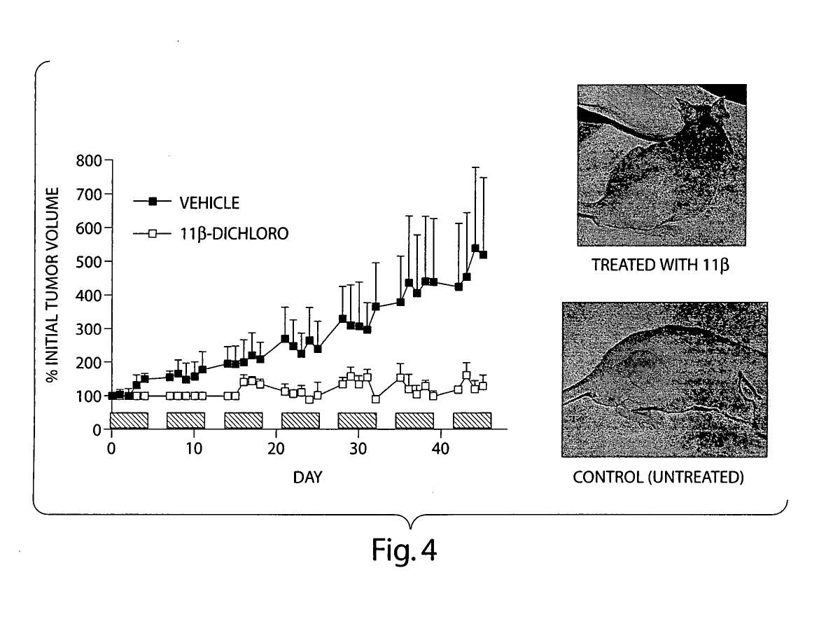

The efficacy of 11β-dichloro was examined toward LNCaP cells grown as a xenograft in nude mice. The compound was shown to be impressively inhibitory to the growth of this tumor. Animals bearing LNCaP xenografts were treated with seven consecutive weekly five-day cycles of 30 mg 11β-dichloro/kg administered via IP injection. This treatment regimen resulted in inhibition of tumor growth as shown in FIG. 4. Inhibition of tumor growth was also obtained with the human colon adenocarcinoma cell line DLD-1 and with an engineered estrogen receptor ligand binding domain-positive HeLa cell line also grown as xenografts in nude mice. The results with these AR-negative cell lines suggest that there are AR dependent as well as independent mechanisms of antitumor action of 11β-dichloro.

Experimental Procedures

Reaction of 11β Compounds with DNA. A self complementary 16-mer oligonucleotide was obtained from IDT DNA, Coralville, Iowa, and was purified by denaturing PAGE. The oligonucleotide was 5′ end labeled with [γ-³²P]ATP and allowed to react with test compounds at 37° C. for 4 h. To determine sites of modification, the adducted oligonucleotide was treated with 1M piperidine for 1 h at 90° C. and fragments were resolved by denaturing PAGE. Reaction products were visualized and quantified by PhosphorImager analysis. The calculated percent cleavage is the proportion of radioactivity in the fragments divided by the total and represents the extent of covalent modification by the test compound.

Relative Affinity of 11β-Compounds for Steroid Receptors. The relative binding affinities (RBA) of 11β compounds for the AR and PR were assessed using a competitive binding assay. Whole cell extracts prepared from LNCaP and T47D cells were used as sources of the AR and PR respectively. RBAs were determined by addition of increasing amounts of unlabeled test compounds to cell extracts in the presence of radiolabeled ligands ([³H]-R1881, 83.5 Ci/mmol, or [³H]-progesterone 103.0 Ci/mmol; NEN, Boston, Mass.). The amount of radiolabeled ligand that remained bound to protein after removal of free ligand by adsorption to dextran-charcoal was determined by scintillation counting.

Relative Affinity of 11β-DNA Adducts for the AR and PR. The identical competitive binding assay was used to investigate the ability of 11β-DNA adducts to bind to the AR and PR. In this case, the covalently modified 16-mer deoxyoligonucleotide prepared as described above was used as a competitor. Following reaction with 11β-dichloro, unreacted compound was removed from the modified 16-mer via three consecutive ethanol precipitations. The absence of unreacted 11β-dichloro was confirmed and the concentration of covalent adducts in the DNA was estimated by conducting a parallel experiment with [¹⁴C]-11β. Increasing amounts of modified or unreacted DNA were added to cell extracts in the presence of radiolabeled ligands. Following incubation, unbound ligand was removed and the amount remaining bound to the receptor determined as described above.

Cell Culture. Cell lines were obtained from the American Type Culture Collection (ATCC; Rockville, Md.). The LNCaP cell line was maintained in RPMI 1640 supplemented with 2.5 mg/ml glucose, 10% fetal bovine serum (FBS; Hyclone, Salt Lake City, Utah), 2 mM glutamax, 1 mM sodium pyruvate and 100 mM HEPES. The T47D line was maintained in MEM-alpha medium containing 10% FBS (Hyclone, Logan, Utah), 0.1 mM non-essential amino acids, 100 mM HEPES, 2 μg/ml bovine insulin, and 1 ng/ml human epidermal growth factor (Invitrogen, Carlsbad, Calif.). Cells were grown in a humidified 5% CO₂/air atmosphere at 37° C. For studies of cell morphology, LNCaP cells were grown on 13 mm diameter Nunc Thermanox cover slips coated with poly-L-lysine (Invitrogen). At indicated time after treatment, cells were washed twice in PBS, fixed in methanol, air dried, and stained with Giemsa.

Cell Cycle Analysis. Cells in exponential growth were treated with test compounds dissolved in DMSO. At the indicated times, drug-containing media was removed and detached cells were collected by centrifugation. Attached cells were harvested by trypsinization, pooled with recovered detached cells, and washed once in PBS. Cells were fixed in 70% ethanol and stored at 4° C. For flow cytometry, cells were resuspended in 0.5 ml of a PBS solution containing 0.1% Triton X-100, 0.2 mg/ml DNase-free RNase, and 0.02 mg/ml propidium iodide (Sigma, St. Louis, Mo.). Cells were analyzed using a Becton Dickinson FACScan flow cytometer with Cell Quest software (MIT Flow Cytometry Core Facility). Data was analyzed using ModFitLT 2.0 software.

Annexin V Staining and Analysis. LNCaP cells in exponential growth were treated with test compounds as described for cell cycle analysis. At indicated times, cells were trypsinized, washed with PBS, and stained with Annexin V-PE and 7-amino-actinomycin D according to manufacturer's protocols (BD-Pharmigen, San Diego, Calif.). Stained cells were analyzed by flow cytometery.

DNA Isolation and Gel Electrophoresis. Adherent cells were scraped directly into growth media and collected along with any detached cells by centrifugation at 0° C. Cells were lysed in a solution containing 50 mM Tris (pH 8.0), 100 μM EDTA, 0.5 mg/ml Proteinase K and 0.5% sodium lauryl sulfate. After incubation at 50° C. for 3 h, the lysates were extracted once with phenol chloroform and nucleic acids were precipitated with ethanol and dissolved in TE pH 7.5. RNA was digested with DNase free RNase (Roche Biochemicals, Indianapolis, Ind.) and the solution was extracted once again with phenol chloroform. DNA was then isolated by ethanol precipitation and the quantity recovered determined by O.D. 260 nm. Equal amounts of DNA from each sample were loaded onto a 1.5% agarose gel containing 0.1 μg/ml ethidium bromide and resolved by electrophoresis. DNA was visualized using a UV transilluminator.

Immunoblot Analysis. After exposure to various compounds for indicated times, LNCaP cells were harvested in medium by scraping, washed once in PBS and suspended in 50 mM Tris pH 7.5, 150 mM NaCl, 1 mM EDTA, 1% NP40, 0.5% Na-deoxychloate, 1 mM Na₃VO₄, 1 mM NaF and protease inhibitor cocktail (P8340; Sigma, St. Louis, Mo.) at 0° C. The cell lysate was centrifuged at 14,000×g for 10 min and supernatants collected for analysis. Protein concentrations were determined by the Bradford dye-binding assay (Bio-Rad Laboratories, Hercules, Calif.). Lysates were combined with SDS-PAGE sample buffer (0.3 M Tris pH 6.8, 2% SDS, 1% 2-mercaptoethanol, 10% glycerol) and equal amounts of protein were resolved by SDS-PAGE, followed by transfer to Immobilon-P membranes (Millipore, Bedford, Mass.). Membranes were blocked with 5% nonfat milk in Tris-buffered saline (0.1% Tween 20, 10 mM Tris pH 7.4, 150 mM NaCl) and probed with antibody against the protein of interest. Antibody complexes formed with horseradish peroxidase-conjugated secondary antibodies were visualized by chemiluminescence (Supersignal West; Pierce, Rockford, Ill.). Antibodies: PARP (06-557; Upstate Biotechnology, Lake Placid, N.Y.); p27 Kip1 (2552; Cell Signaling Technologies, Beverly, Mass.); p21 (sc-397; Santa Cruz Biotechnology, Santa Cruz, Calif.); p45 Skp2 (32-3300; Zymed Laboratotries, South San Francisco, Calif.).

Animal Studies. Four to six week old NIH Swiss nu/nu athymic male mice (25 gm) were obtained from the National Cancer Institute-Frederick Cancer Center (Frederick, Md.). Experiments were carried out under guidelines of the MIT Animal Care Committee. Animals were injected subcutaneously in the right flank with 5×10⁶ LNCaP cells suspended in a solution of 50% PBS/50% Matrigel (Collaborative Research, Bedford, Mass.). Therapy commenced when a palpable tumor of approximately 4×4 mm formed (n=5 per treatment group). The 11β-dichloro compound was dissolved in a vehicle composed of cremophor EL, saline and ethanol (43:30:27). Tumor dimensions were measured with vernier calipers. Tumor volumes were calculated using the formula: π/6×larger diameter×(smaller diameter)². Statistical analyses were performed using a paired t-test. At the end of the study period, animals were euthanized with CO₂. At the time of sacrifice, blood samples were taken from several animals in each group for a complete blood count, along with serum chemistry and liver function analyses. A complete necropsy was also performed, including histopathology on two animals from each group.

### Example 4

### Linkers and 7α Compounds

Methods of the invention can also be in combination with 7α-estradiol compounds (7α compounds are illustrated in FIGS. 12-14).

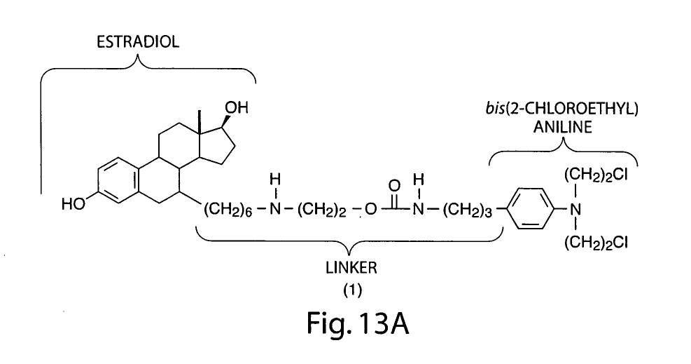

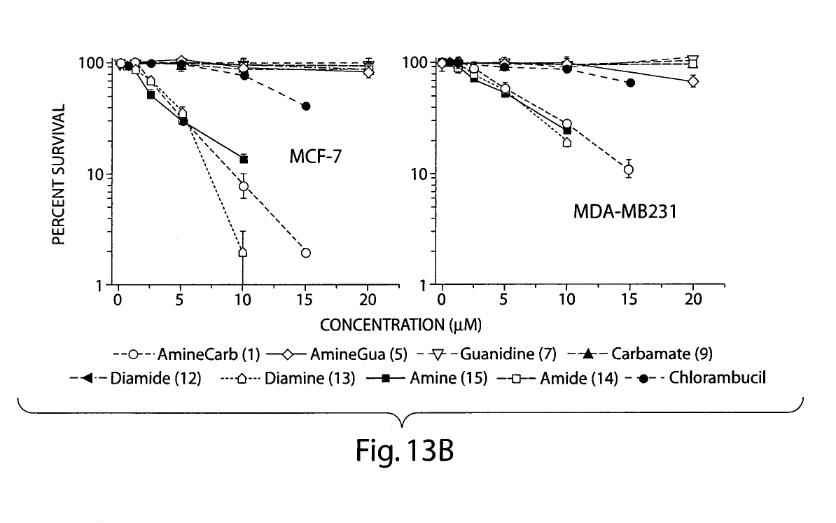

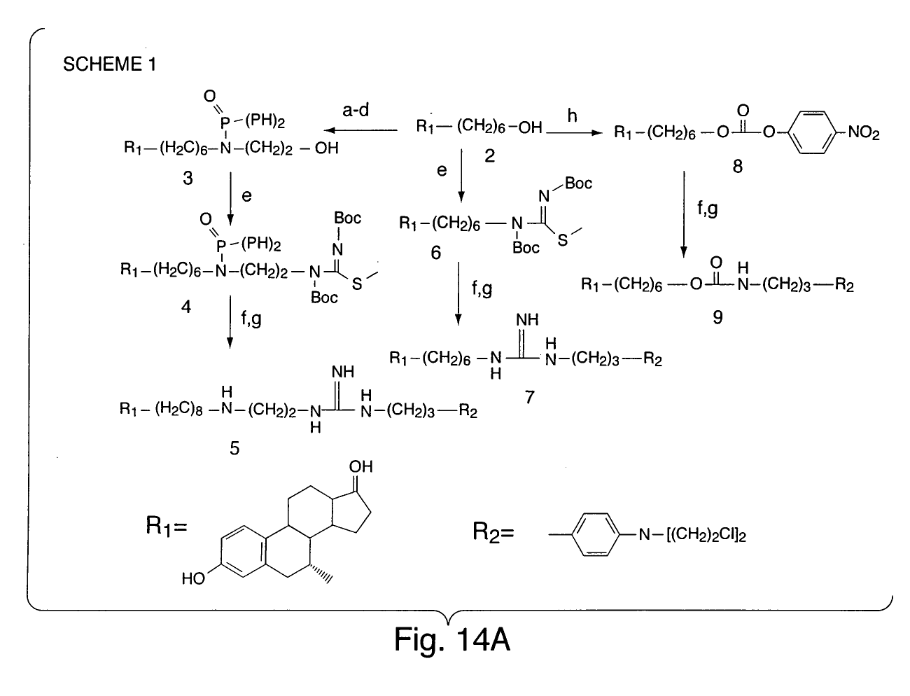

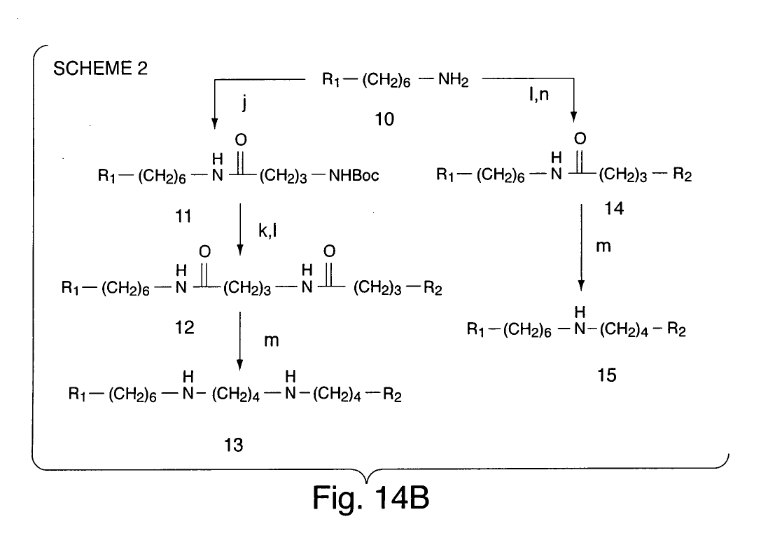

The synthesis of compound 1 (FIG. 13-A) has been described previously. The synthetic steps for the new compounds are shown in Schemes 1 and 2 (FIG. 14). The syntheses utilized 3,17-bis(2-tetrahydropyranyloxy)-7α-(6-hydroxyhexan-1-yl)-estra-1,3,5(10)triene 2 as the starting compound; its preparation has also been described. Construction of linkers proceeded by linear additions to 2 with final addition of the N,N-bis(2-chloroethyl)aniline moiety. Compound 5 was prepared by conversion alcohol 2 to the bromide, which was allowed to react with a protected ethanolamine providing 3. The Mitsunobo reaction then was used to couple 1,3-bis(tert-butoxycarbonyl)-2-methyl-2-thiopseudourea with 3. Reaction of the resulting product 4 with excess (N,N-bis-2-chloroethylaminophenyl)-propylamine followed by acid deprotection produced 5. Procedures described by Linney et al. (J. Med. Chem., 2000, 43, 2362-2370) were applied to incorporate an N,N-disubstituted guanidine moiety into the linker. The preparation of 7 proceeded with the initial reaction of 2 with 1,3-bis(tert-butoxycarbonyl)-2-methyl-2-thiopseudourea under Mitsunobo conditions. The resulting product 6 was then allowed to react with an excess of (N,N-bis-2-chloroethylaminophenyl)-propylamine followed by acid deprotection to furnish 7. Compound 9 was prepared by conversion of 2 to the p-nitrophenyl carbonate 8, which was then allowed to react with (N,N-bis-2-chloroethylaminophenyl)-propylamine. Removal of THP groups under acidic conditions produced 9. Compound 12 containing two amide groups was synthesized by first reacting 10 with the NHS ester of 4(tertbutoxycarbonylamino)butyric acid. Following removal of the THP and Boc groups the terminal amino group was allowed to react with the NHS ester of chlorambucil producing 12. Compound 13 in which the linker contains two amino groups was produced by reduction of 12 with borane dimethylsulfide complex. The preparation of 14 was accomplished by conversion of 2 to the phthalimide via Mitsunobo conditions with subsequent hydrazinolysis to obtain the amine 10. The NHS ester of chlorambucil was then allowed to react with the terminal amine, producing 14. Compound 15, containing a secondary amino group in the linker, was prepared by reduction of the amide in 14 using borane dimethylsulfide complex.

Synthesis and Evaluation of Estradiol-Linked Genotoxicants

The effects of molecular variations in the linker were initially characterized on the two activities that we intended to optimize—affinity for the ER and covalent reaction with DNA. We then examined the ability of DNA adducts produced by each compound to form complexes with a portion of the ER containing the ligand binding site. The lipophilicity (logP and logD) of each molecule was evaluated to obtain estimates of solubility and permeability. Finally, we assessed the toxicity of the new compounds toward ER+ and ER− breast cancer cells to compare the toxicities of each molecule to 1.

In the initial characterization of the biochemical properties of new compounds 5, 7, 9, 12-15 we evaluated their affinities for the ER. A radiometric competitive binding assay with the rabbit uterine ER was used to determine the relative binding affinity (RBA) of each compound for the ER as compared with estradiol where the RBA for estradiol is 100. All of the new compounds retained the hexanyl portion of the linker attached to the 7 position of estradiol. Previous studies established the importance of an alkanyl chain of at least six carbons for binding of modified ligands to the ER. All of the compounds exhibited some affinity for the ER. Some of the synthesized compounds have RBA values for the ER ranging from 6 to 40, with four compounds with RBAs that are comparable to 1. Among the new compounds, 15 containing a single amino group in the linker had the highest affinity for the ER; RBA=40.

Although it is apparent that the original combination of the positively charged secondary amine with the neutral carbamyl group (compound 1) results in a bifunctional compound with excellent affinity for the rabbit uterine ER, compounds 7, 12 and 13 have comparable affinities. These molecules were viewed as valuable assets as we move ahead toward probing structure-activity relationships and the biochemical mechanisms underlying the biological activity of 1. It is likely that our 7α-linked estradiol compounds adopt a binding mode similar to that identified for the 7α-undecylamide estradiol analog ICI 164,384. The positioning and orientation of the estradiol moiety of ICI-164,384 within the hydrophobic binding cavity of the ER is directed by its 7α side chain, which protrudes out of a hydrophobic channel extending from the binding pocket. At the surface of the LBD, a 90° flexion of the undecyl chain enables the remainder of the linker to track closely with the surface contours of the LBD 10. The low RBAs of compounds 5, 9 and 14 may result from surface interactions adopted by the linkers in these molecules that create a misalignment of the estradiol moiety within the binding cavity. The ability of the bis-(2-chloroethyl)-aniline moiety of our bifunctional compounds to react covalently with DNA is requisite for our intended mechanism of action. The reactivity of each compound with DNA was assessed by its ability to produce piperidine labile sites in the self complementary octamer deoxyoligonucleotide (data not shown). Compound 9 in which the alkyl linker contains a single carbamyl group produced the lowest level of modification (i.e., 3% cleaved by piperidine). Compound 14 containing an amido instead of the carbamyl group produced approximately five times the number of DNA adducts (14% cleaved by piperidine). High levels of reactivity towards DNA were observed with compounds with linkers containing secondary amino groups. For example, compound 13 in which the linker contains a diamine—NH—(CH2)4-NH—CH2- was the most reactive (79% cleaved by piperidine). Addition of an amino group to the linker in the least reactive compound 9 produced the aminocarbamyl linked compound 1. The reactivity of 1 was similar to that of 15 in which the linker contains a single secondary amine suggesting that the charged amino group is the major determinant of reaction rate. The same is likely the case for molecules 5 and 7 in which the strongly basic guanidino groups would be cationic under assay conditions. It is likely that the cationic nature of these molecules gives them a high reactivity with DNA by localizing the reactive alkylating group in the vicinity of nucleophilic atoms. A similar result has been reported for a conjugate of chlorambucil with the polyamine spermidine. Using an electrophoretic gel mobility shift assay, we observed that covalent DNA adducts of 1, 5, 7, 13 and 15 form complexes with the portion of the ER containing the estradiol binding site. Under conditions that allowed complex formation, addition of the ER to the modified DNAs resulted in the appearance of a slowly migrating band by electrophoresis that was eliminated by addition of excess competitor, estradiol (data not shown). The the extent of complex formation for 1, 7, 13 and 15 were correlated with the RBAs of the unreacted compounds. The exception was compound 5 in which the linker contained both amino and guanidino groups. In this case, despite its low RBA, virtually all of the modified oligonucleotide formed a slowly migrating band. We do not know the basis for this unexpected finding. LogP and logD values can be predictive of aqueous solubility, absorbtion and permeability. The lipophilicities of 1, 5, 7, 9, 12-15 were assessed using an HPLC method to estimate the logP of the neutral form of each compound. LogD values at pH 7.4 were estimated using an equation derived by Horvath et al. 13 for basic compounds (data not shown). The logD values indicated that the aqueous solubilities of the eight compounds span approximately a 2,500-fold range under physiological conditions. The compounds with logD values >5 (compounds 9 and 14) had both low affinities for the ER and low reactivity with DNA. Compounds containing charged groups with calculated logD values <3 generally had the highest affinities for the ER along with the greatest reactivities towards DNA. These relationships, however, did not prove to be reliable predictors of biological activities in cytotoxicity assays against breast cancer cells. Changes in linker structure had a significant effect on toxicity. The lethal effects of our new compounds were investigated in the MCF-7 (ER+) and MDA-MB231 (ER−) breast cancer cell lines. The data shown in FIG. 13 indicate that most but not all of the modifications that were introduced in the linker resulted in decreased toxicity towards both cell lines. The low toxicity of 5 and 7, which contain guanindinium groups, may be related to either their poor uptake by cells or their rapid excretion once absorbed. Despite showing reactivity towards DNA in vitro, neither compound showed significant toxicity at the highest dose; i.e., 20 μM. Lack of uptake may also be responsible for the low toxicity of 9, 12 and 14, which have high logD values that are not predictive of good absorption. Further work is warranted to determine if cellular uptake is indeed limiting for these compounds. As previously reported, 1 was significantly more toxic toward MCF-7 cells than MDA-MB231 cells.6 Compounds 13 and 15 containing amino groups showed toxicity similar to that of 1. Both of these compounds also showed greater toxicity toward the ER-positive MCF-7 cells than towards the ER-negative MDA-MB231 cells. This result was consistent with our intended mechanisms, since the RBAs and reactivities with DNA of 13 and 15 imply greater toxicity on ER-positive cells. It is interesting that the results of the electrophoretic mobility shift assay indicate that DNA adducts of 1 have the greatest affinity for the ER-LBD; compound 1 also shows the largest differential toxicity between the two cell lines.

Having now described some illustrative embodiments of the invention, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the invention. In particular, although many of the examples presented herein involve specific combinations of method acts or system elements, it should be understood that those acts and those elements may be combined in other ways to accomplish the same objectives. Acts, elements and features discussed only in connection with one embodiment are not intended to be excluded from a similar role in other embodiments. Further, for the one or more means-plus-function limitations recited in the following claims, the means are not intended to be limited to the means disclosed herein for performing the recited function, but are intended to cover in scope any means, known now or later developed, for performing the recited function. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All patents and patent publications (including U.S. Pat. Nos. 5,879,917; 5,882,941; and 6,500,669), references and other publications, including kit protocols that are recited in this application are incorporated in their entirety herein by reference.

What is claimed is:

1. A method for killing androgen receptor negative cells by contacting the cells with an effective amount of a compound, wherein the compound comprises:
a bifunctional DNA damaging moiety
linked by a linker that is stable under intracellular conditions
to a ligand for an androgen receptor.

2. A method for killing estrogen receptor negative cells by contacting the cells with an effective amount of a compound, wherein the compound comprises:
a bifunctional DNA damaging moiety
linked by a linker that is stable under intracellular conditions
to a ligand for an estrogen receptor.

3. A method for killing vitamin D receptor negative cells by contacting the cells with an effective amount of a compound, wherein the compound comprises:
a bifunctional DNA damaging moiety
linked by a linker that is stable under intracellular conditions
to a ligand for a vitamin D₃ receptor.

4. The method of claim 1 wherein the ligand is estradienone.

5. The method of claim 1 wherein the compound is 11β-{N-(2-(N-((N,N-bis-2-chloroethylaminophenyl)propyl)-carbamoyloxy) ethyl)aminohexyl}-17β-hydroxy-estra-Δ4(5),9(10)-3-one.

6. The method of claim 1 wherein the cells are resistant to DNA damaging agents.

7. The method of claim 1 wherein the cells are selected from the list of cancer cells consisting of: breast, ovarian, endometrial, colon, melanoma, lymphoma and pancreatic cancer.

8. The method of claim 2 wherein the ligand is 2-phenyl-indole.

9. The method of claim 2 wherein the ligand is estradiol.

10. The method of claim 2 wherein the compound is 1-6{N-[2-(N-((N,N-bis-2-chloroethylaminophenyl)propyl)-carbamoyl oxy)ethyl]aminohexyl}-5-hydroxy-2-(4-hydroxyphenyl)-3-methyl indole.

11. The method of claim 2 wherein the compound is 7α-{N-(2-(N-((N,N-bis-2-chloroethylaminophenyl)propyl)-carbamoyloxy) ethyl)aminohexyl}-3,17β-dihydroxyestra-1,3,5(10)-triene.

12. The method of claim 2 wherein the cells are resistant to DNA damaging agents.

13. The method of claim 2 wherein the cells are selected from the list of cancer cells consisting of: prostate, colon, melanoma, lymphoma and pancreatic cancer.

14. The method of claim 3 wherein the ligand is vitamin D₃.

15. The method of claim 3 wherein the compound is (3-{4-[Bis-(2-chloro-ethyl)-amino]-phenyl}-propyl)-carbamic acid 2-[3-(4-{4-[2-(3,5-dihydroxy-2-methylene-cyclohexylidene)-ethylidene]-7α-methyl-octahydro-inden-1-yl}-8-hydroxy-8-methyl-nonyloxy)-propylamino]-ethyl ester.

16. The method of claim 3 wherein the cells are resistant to DNA damaging agents.

17. The method of claim 3 wherein the cells are selected from the list of cancer cells consisting of: breast, ovarian, lymphoma and endometrial cancer.

18. A cell membrane permeant compound effective in inducing cell cycle arrest comprising
a non-alkylating aniline moeity
linked by a linker that is stable under intracellular conditions and
an agent that mediates binding of a cellular protein to the compound.

19. The compound of claim 18 wherein the agent is a ligand for a steroid or secosteroid receptor.

20. The compound of claim 19 wherein the ligand is selected from a group consisting of: estradienone, estradiol, 2-phenylindole and vitamin D₃.

21. A cell membrane permeant compound effective in inducing cell cycle arrest comprising
a monofunctional DNA alkylating aniline moiety
linked by a linker that is stable under intracellular conditions and
an agent that mediates binding of a cellular protein to the compound.

22. The compound of claim 21 wherein the agent is a ligand for a steroid or secosteroid receptor.

23. The compound of claim 22 wherein the ligand is selected from a group consisting of: estradienone, estradiol, 2-phenylindole and vitamin D₃.

24. A method for inducing cell cycle arrest by administering a sufficient amount of the compound of claim 18 or 21 to induce cell cycle arrest.

25. The method of claim 14, where in the compound is 11β-{N-(2-(N-((N,N-bis-2-chloroethylaminophenyl)propyl)-carbamoyloxy) ethyl)aminohexyl}-17β-hydroxy-estra-Δ4(5),9(10)-3-one.

26. A method for treating cancer in patient in need thereof, comprising administering to the patient a compound of claim 18 or 21 in combination with another anti-cancer agent.

27. A method for treating a patient with androgen receptor negative cancer by administering to the patient a therapeutically effective amount of a compound, wherein the compound comprises:
a bifunctional DNA damaging moiety
linked by a linker that is stable under intracellular conditions
to a ligand for an androgen receptor.

28. A method for treating a patient with estrogen receptor negative cancer by administering to the patient a therapeutically effective amount of a compound, wherein the compound comprises:
a bifunctional DNA damaging moiety
linked by a linker that is stable under intracellular conditions
to a ligand for an estrogen receptor.

29. A method for treating a patient with vitamin D receptor negative cancer by administering to the patient a therapeutically effective amount of a compound, wherein the compound comprises:
a bifunctional DNA damaging moiety
linked by a linker that is stable under intracellular conditions
to a ligand for a vitamin D receptor.

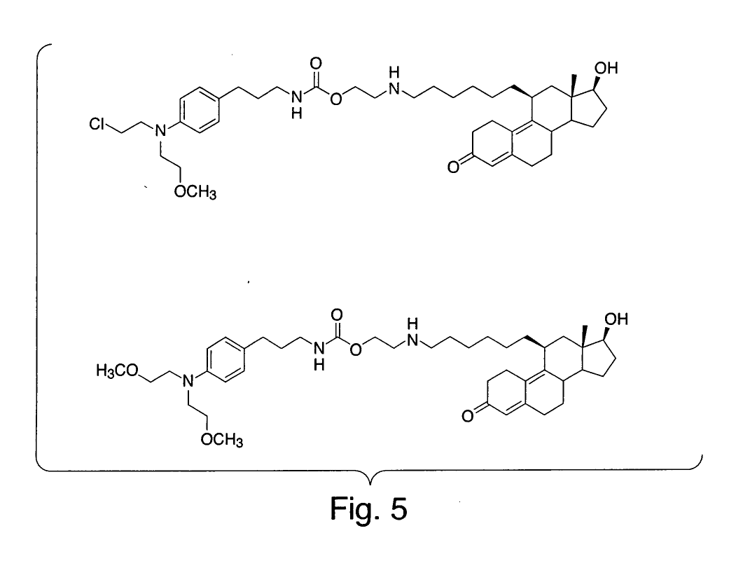

30. The method of claim 1 or 27, wherein the compound has a formula selected from the group consisting of the formulas shown in FIGS. 1 and 5.

31. A pharmaceutical composition comprising a compound of claim 18 or 21, or a stereoisomeric form, or a pharmaceutically acceptable acid or base addition salt form thereof.

32. The compounds of claim 18 or 21 wherein the linker comprises an alkyl-amino-carbamate alkyl chain.

33. The compounds of claim 18 or 21 wherein the alkyl chain has six carbons.

34. A method for treating cancer comprising
determining the level of Skp2 expression in the cancer cell
and if the level of Skp2 is increased
contacting the cancer cell with an effective amount of a compound, wherein the compound comprises:
a bifunctional DNA damaging moiety
linked by a linker that is stable under intracellular conditions
to a ligand for a steroid or secosteroid receptor.

35. A method for treating cancer comprising
determining the level of c-Myc expression in the cancer cell
and if the level of c-Myc is increased
contacting the cancer cell with an effective amount of a compound, wherein the compound comprises:
a bifunctional DNA damaging moiety
linked by a linker that is stable under intracellular conditions
to a ligand for a steroid or secosteroid receptor.

36. A method for treating cancer comprising
determining the level of phosphorylation of p70S6K in the cancer cell
and if the level of phosphorylation of p70S6K is increased
contacting the cancer cell with an effective amount of a compound, wherein the compound comprises:
a bifunctional DNA damaging moiety
linked by a linker that is stable under intracellular conditions
to a ligand for a steroid or secosteroid receptor.

37. A method for treating cancer comprising
determining the level of Bcl-2 expression in the cancer cell
and if the level of Bcl-2 is increased
contacting the cancer cell with an effective amount of a compound, wherein the compound comprises:
a bifunctional DNA damaging moiety
linked by a linker that is stable under intracellular conditions
to a ligand for a steroid or secosteroid receptor.
